(19) United States
(12) Patent Application Publication
(10) Pub. No.: US 2005/0239082 A1
(43) Pub. Date: Oct. 27, 2005

(54) Alien sequences

(76) Inventors: Temple Smith, Marblehead, MA (US); Prashanth Vishwanath, Allston, MA (US); Sean Quinlan, Melrose, MA (US)

Correspondence Address: CHOATE, HALL & STEWART LLP, TWO INTERNATIONAL PLACE, BOSTON, MA, 02110, US

(21) Appl. No.: 10/763,039

(22) Filed: Jan. 22, 2004

Related U.S. Application Data
(60) Provisional application No. 60/441,832, filed on Jan. 22, 2003.

(51) Int. Cl. C12Q 1/68; C12M 1/34

(52) U.S. Cl. 435/6; 435/287.2

(57) ABSTRACT

The present invention provides sequences and reagents for preparing microarrays with internal controls. Specifically, the present invention defines and provides sequences that are not present in the hybridizing mRNA or cDNA, and therefore can be used both as hybridization controls and for inter-spot normalization.

### RELATED APPLICATION

This application claims priority to Provisional Patent Application No. 60/441,832, filed Jan. 22, 2003, which is incorporated herein by reference in its entirety.

### BACKGROUND

The proper and harmonious expression of a large number of genes is a critical component of normal growth and development and the maintenance of proper health. Disruptions or changes in gene expression are responsible for many diseases. Using traditional methods to assay gene expression, researchers were able to survey a relatively small number of genes at a time. Microarrays allow scientists to analyze expression of many genes in a single experiment quickly and efficiently. A microarray works by exploiting the ability of a given mRNA molecule to bind specifically to, or hybridize to, the DNA template from which it originated.

DNA arrays are commonly used to study gene expression. In this type of study, mRNA is extracted from a sample (for example, blood cells or tumor tissue), converted to complementary DNA (cDNA) and tagged with a fluorescent label. In a typical microarray experiment, cDNA from one sample (sample A) is labeled with a first dye that fluoresces in the red and cDNA from another sample (sample B) is labeled with a different dye that fluoresces in the green. The fluorescent red and green cDNA samples are then applied to a microarray that contains DNA fragments (oligonucleotides) corresponding to thousands of genes. If a DNA sequence probe is present on the microarray and its complement is present in one or both samples, the sequences bind, and a fluorescent signal can be detected at the specific spot on the array, where the DNA sequence probe is located. The signals are generally picked up using a “scanner” which creates a digital image of the array. The red to green fluorescence ratio in each spot reflects the relative expression of a given gene in the two samples. The result of a gene expression experiment is referred to as a gene expression “profile” or “signature”.

This technology, though widely used, is not without its problems. Almost every procedure in the methodology is a potential source of fluctuation leading to a lot of noise in the system as a whole. The major sources of fluctuations to be expected are in mRNA preparation, reverse transcription leading to cDNA of varying lengths, systemic variation in pin geometry, random fluctuations in spot volume, target fixation, slide non-homogeneities due to unequal distribution of the probe, hybridization parameters and non-specific hybridization. Some of the errors mentioned above can be minimized by performing replicates of experiments or by using a flipped dye design.

Biological replicates are arrays that each use RNA samples from different individual organisms, pools of organisms or flasks of cells, but yet compare the same treatments or control/treatment combinations. Technical replicates are arrays that each use the same RNA samples and also the same treatment. Thus, in this setting, the only differences in measurements are due to technical differences in array processing. The rationale for the flipped dye design is that it allows for the estimation and removal of gene specific dye effects. These dye effects have been shown to be reproducible across independent arrays by the use of Control vs. Control arrays. Any deviation from a ratio of 1 in these arrays is due to either dye effect or residual error. However, none of these methods will account accurately for chip manufacturing error.

Therefore, there remains a need for the development of improved microarray technologies, and particularly technologies that allow researchers to control for errors and/or to normalize signals.

### SUMMARY OF THE INVENTION

The present invention provides reagents and methods that are useful in normalizing and standardizing data from nucleic acid hybridization studies, and particularly from microarray-based hybridizations. The present invention teaches that it is useful to define nucleotide sequences that are “alien” to the sequence population under analysis. Such alien sequences may be included on microarrays and will not hybridize with the nucleic acid population under study. Alternatively or additionally, sequences complementary to the alien sequences may be mixed together with (i.e., “spiked” into) the hybridizing population in order to control for processing and hybridization events.

Use of the alien sequences (and/or their complements) according to the present invention provides a number of advantages. For instance, when an alien sequence is included in a microarray and its complement is not included in the hybridizing sample, the alien sequence may act as a negative control, revealing defects in hybridization conditions that could affect the experimental outcome.

Furthermore, when an alien oligonucleotide is present on an array, its complement may be added to the hybridizing sample, and processed and hybridized together with that sample, as a control for the processing/hybridization steps. If the alien oligonucleotide is present in spots at different locations on the chip, this strategy can also be used to control intra-chip hybridization variations.

Moreover, when the amount of anti-alien spiking nucleic acid (and/or alien oligonucleotide) is known in advance, the degree of anti-alien/alien hybridization may be relied upon to establish the amount of non-alien sequences present in the hybridizing sample based on the relative extent of their hybridization to complementary oligonucleotides. In fact, in some embodiments, multiple alien/anti-alien pairs at different amounts are utilized in order to provide multiple points for comparative quantitation of other nucleic acids. In certain preferred embodiments, the alien sequence probe and the probe detecting the target sequence to be quantified are mixed together in the same spot to allow in situ comparisons. This approach also provides a consistent standard (the fixed amount of alien probe) that can be relied upon to allow inter-slide comparisons and inter-experiment comparisons even when the experiments are carried out using rare samples (i.e., in a situations where the number of experimental replicates that can be performed for control purposes is limited), or over long time spans, etc.

Thus, alien sequence probes and their complements can be used to normalize the data obtained from array hybridizations. For instance, if every spot in an array contains a defined ratio of experimental probes to alien probes, the presence of the alien probes allows the researcher to control for variations between or among spots (e.g., by hybridizing the array with a sample containing anti-alien sequences that are differently labeled from the nucleic acid sequences under study).

Additionally, the presence of alien probes in microarray spots allows researchers to assess the quality and consistency of microarray fabrication and/or priting/spotting techniques. For example, when alien sequences are present in all or a representative collection of spots, the presence or absence of particular spots, overall spot morphology, and slide quality can often be assessed by hybridization (in parallel or simultaneously with experimental hybridization) with an anti-alien nucleic acid. Even random spotting of alien sequences can provide information about the overall integrity or uniformity of a slide. Often, however, it will at least be desirable to include alien sequences in one or more spots containing experimental samples so as to provide a direct assessment of an experimentally relevant spot.

### DESCRIPTION OF THE DRAWING

FIG. 1 shows 100 sequences identified according to the present invention as “alien” to mouse cDNA.

FIG. 2 shows about 50 oligonucleotides identified according to the present invention as alien to mouse cDNA and useful for hybridization applications.

FIG. 3 shows that inventive alien oligonucleotides, selected as alien to both mouse and human cDNAs, do not hybridize with commercially available universal mouse and human mRNA sets. The presence of alien oligonucleotide probes on the slide is demonstrated on FIG. 3A, by detection of fluorescent signals over the whole array, after enzymatic 3′-OH labeling with terminal deoxynucleotidyl transferase in the presence of dCTP-Cy3. FIG. 3B shows that in the absence of such treatment the alien probe sequences failed to yield appreciable signal intensity above background threshold, while the human and mouse positive control sequence probes were detectable.

FIG. 4 ranks the alien oligonucleotides depicted in FIG. 2 based on normalized median fluorescence intensity minus background when hybridized with standard human and mouse mRNA samples.

FIG. 5 ranks the alien oligonucleotides depicted in FIG. 2 based on their percentage of hybridization with standard human and mouse mRNA samples, as compared with the positive control oligonucleotides designed to hybridize with those samples.

FIG. 6 illustrates the inventive anti-alien in-spike control concept. Panels A-C show sequences of alien genes designed by linking four 70mer alien sequences together. Panel D shows a microarray containing four alien oligonucleotides whose sequences are present in one of the alien genes, and four that are unrelated. Panel E shows that cDNAs corresponding to the non-coding strand of the alien gene hybridize with the expected alien oligonucleotides on the chip, and not with the unrelated alien oligonucleotides.

FIG. 7 illustrates the inventive concept of using alien sequences as internal controls for microarray spotting and hybridization. Microarrays were constructed in which a single alien oligonucleotide, A0892, was spotted by itself or with a mixture of other 70mer oligonucleotide probes. A0892 alone or the probe mixture containing A0892 were spotted in concentrations ranging from 2 to 20 μM. The figure insert presents a small area of such a microarray. The graph shows the variations of the normalized signal intensity as a function of concentration of probe mixture, for A0892-alone spots and mixture spots.

FIG. 8 illustrates the inventive concept of using an alien oligonucleotide and its complementary sequence as controls for in situ normalization. In such experiments, a microarray, to which an alien 70mer probe has been co-printed with different gene specific probes, is contacted with an hybridization mixture containing the complementary sequence of the alien oligo labeled with Alexa-488, and two different nucleic acid test samples labeled with Cy3 and Cy5, respectively. A 3 color laser scanner is used to analyze the hybridized microarray.

### DEFINITIONS

Throughout the specification, several terms are employed, that are defined in the following paragraphs.

Alien gene—As used herein, the term “alien gene” refers to a nucleotide molecule comprised of at least two concatermerized alien sequences. The gene may contain multiple copies of a single alien sequence, or alternatively may contain a plurality of different alien sequences. An alien gene may be single or double stranded, and may contain or be associated with a promoter or other control sequence that will direct the production of a template of either strand of the gene. In particular, as will be clear from discussions herein, in some embodiments of the invention it will be desirable to produce an alien gene transcript that is an alien sequence, whereas in other embodiments it will be desirable to produce an alien gene transcript that is complementary to an alien sequence.

Alien sequence—A nucleotide sequence is considered “alien” to a particular source or collection of nucleic acids if it does not hybridize with nucleic acids in the source or collection. For example, if the source or collection is mRNA from normal kidney cells, an oligonucleotide will have a sequence that is “alien” to the mRNA if its complement is not present in the mRNA. Conversely, if the source or collection is cDNA from the same cells, then an oligonucleotide will have a sequence that is “alien” to the cDNA if its complement is not present in the cDNA. In certain preferred embodiments of the invention, the source or collection comprises expressed nucleic acids (e.g., mRNA or cDNA) of a target organism (e.g., mouse, dog, human, etc), tissue (e.g., breast, lung, colon, liver, brain, kidney, etc), or cell type (e.g., before or after exposure to a particular stimulus or treatment). Alternatively or additionally, the source or collection may preferably be a plurality of nucleic acids to be hybridized to an array.

Hybridizing sample—The terms “hybridizing sample” and “hybridizing mixture” are used herein interchangeably. They refer to the nucleic acid sample being or intended to be hybridized to a microarray. Those of ordinary skill in the art will appreciate that the hybridizing sample may contain DNA, RNA, or both, but most commonly contains cDNA. Those of ordinary skill in the art will further appreciate that the hybridizing sample typically contains nucleic acids whose hybridization with probes on an array is detectable. For example, in many embodiments, the hybridizing sample comprises or consists of detectably labeled nucleic acids.

Detectably labeled—The terms “labeled”, “detectably labeled” and “labeled with a detectable agent” are used herein interchangeably. They are used to specify that a nucleic acid molecule or individual nucleic acid segments from a sample can be detected and/or visualized following binding (i.e., hybridization) to probes immobilized on an array. Nucleic acid samples to be used in the methods of the invention may be detectably labeled before the hybridization reaction or a detectable label may be selected that binds to the hybridization product. Preferably, the detectable agent or moiety is selected such that it generates a signal which can be measured and whose intensity is related to the amount of hybridized nucleic acids. Preferably, the detectable agent or moiety is also selected such that it generates a localized signal, thereby allowing spatial resolution of the signal from each spot on the array. Methods for labeling nucleic acid molecules are well known in the art (see below for a more detailed description of such methods). Labeled nucleic acids can be prepared by incorporation of or conjugation to a label, that is directly or indirectly detectable by spectroscopic, photochemical, biochemical, immunochemical, radiochemical, electrical, optical, or chemical means. Suitable detectable agents include, but are not limited to: various ligands, radionuclides, fluorescent dyes, chemiluminescent agents, microparticles, enzymes, colorimetric labels, magnetic labels, and haptens. Detectable moieties can also be biological molecules such as molecular beacons and aptamer beacons.

Fluorescent Label—The terms “fluorophore”, “fluorescent moiety”, “fluorescent label”, “fluorescent dye” and “fluorescent labeling moiety” are used herein interchangeably. They refer to a molecule which, in solution and upon excitation with light of appropriate wavelength, emits light back. Numerous fluorescent dyes of a wide variety of structures and characteristics are suitable for use in the practice of this invention. Similarly, methods and materials are known for fluorescently labeling nucleic acids (see, for example, R. P. Haugland, “Molecular Probes: Handbook of Fluorescent Probes and Research Chemicals 1992-1994”, 5^{th} Ed., 1994, Molecular Probes, Inc.). In choosing a fluorophore, it is generally preferred that the fluorescent molecule absorbs light and emits fluorescence with high efficiency (i.e., it has a high molar absorption coefficient and a high fluorescence quantum yield, respectively) and is photostable (i.e., it does not undergo significant degradation upon light excitation within the time necessary to perform the array-based hybridization). Suitable fluorescent labels for use in the practice of the methods of the invention include, for example, Cy-3™, Cy-5™, Texas red, FITC, Spectrum Red™, Spectrum Green™, Alexa-488, phycoerythrin, rhodamine, fluorescein, fluorescein isothiocyanine, carbocyanine, merocyanine, styryl dye, oxonol dye, BODIPY dye, and equivalents, analogues or derivatives of these molecules.

Microarray—The terms “microarray”, “chip” and “biochip” are used herein interchangeably. They refer to an arrangement, on a substrate surface, of multiple nucleic acid molecules of known or unknown sequences. These nucleic acid molecules are immobilized to discrete “spots” (i.e., defined locations or assigned positions) on the substrate surface. A discrete spot may contain a single nucleic acid molecule or a mixture of different nucleic acid molecules. Spots on an array may be arranged on the substrate surface at different densities. In general, microarrays with probe pitch smaller than 500 μm (i.e., density larger than 400 probes per cm²) are referred to as high density microarrays, otherwise, they are called low density microarrrays. Arrays come as two-dimensional probe matrices (or supports), which can be solid or porous, planar or non-planar, unitary or distributed. The term “micro-array” more specifically refers to an array that is miniaturized so as to require microscopic examination for visual evaluation. Arrays used in the methods of the invention are preferably microarrays. The present invention provides microarrays in which at least one spot contains an alien oligonucleotide. Other types of microarrays and sets of microarrays provided by the invention are described below.

Oligonucleotide—As used herein, the term “oligonucleotide”, refers to usually short strings of DNA or RNA to be used as hybridizing probes or nucleic acid molecule array elements. These short stretches of sequence are often synthesized chemically. As will be appreciated by those skilled in the art, the length of the oligonucleotide (i.e., the number of nucleotides) can vary widely, often depending on its intended function or use. Generally, oligonucleotides of at least 6 to 8 bases are used, with oligonucleotides ranging from about 10 to 500 bases being preferred, with from about 20 to 200 bases being particularly preferred, and 40 to 100 bases being especially preferred. Longer oligonucleotide probes are usually preferred in array-based hybridization reactions, since higher stringency hybridization and wash conditions can be used, which decreases or eliminates non-specific hybridization.

Probe—For the purposes of the present invention, a “probe” is an nucleic acid, often an oligonucleotide that is, or is intended to be, attached to a solid support in an array. Preferably, the rpobes that comprise a microarray or biochip are of a defined length and similarity. This allows for similar hybridization characteristics. As is well known to those skilled in the art, for the hybridization characteristics to be similar across a wide range of oligonucleotides, it is typically required that the probes on the array be of the substantially same length, have a similar percentage of Guanine to Cytosine content and lack any extensive runs of poly A, poly G, poly C, or poly T tracts. The goal of controlling these parameters is to produce probes that have similar melting and hybridization temperatures. Additionally, these probes should, preferably, lack length complementary regions and not form hairpin structures.

Target—The term “target” refers to nucleic acids intended to be hybridized (or bound) to probes immobilized on microarrays by sequence complementarity. As is well-known in the art, target nucleic acids may be obtained from a wide variety of organisms, tissues or cells. Methods and techniques for the extraction, manipulation and preparation of nucleic acids for hybridization reactions are well-known in the art (see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons).

Hybridization—The term “hybridization” has herein its art understood meaning and refers to the binding of two single stranded nucleic acids via complementary base pairing. A hybridization reaction is called specific when a nucleic acid molecule preferentially binds, duplexes, or hybridizes to a particular nucleic acid sequence under stringent conditions (e.g., in the presence of competitor nucleic acids with a lower degree of complementarity to the hybridizing strand).

High stringency conditions—For microarray-based hybridization, standard “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for at least 8 hours, followed by one or more washes in 0.2×SSC, 0.1% SDS at 65° C. “Moderate stringency conditions” are defined for solution phase hybridization as aqueous hybridization (i.e., free of formamide) in 6×SSC, 1% SDS at 65° C. for at least 8 hours, followed by one or more washes in 2×SSC, 0.1% SDS at room temperature.

### Description of Certain Preferred Embodiments of the Invention

The present invention provides reagents and methods that are useful in normalizing and standardizing data from nucleic acid hybridization studies, and particularly from microarray hybridizations. The present invention teaches that it is useful to define nucleotide sequences that are “alien” to the sequence population under analysis.

In particular, the use of such alien oligonucleotide sequences in micro-array based hybridization is herein described to be able to serve several distinct control purposes. For example, (1) when spotted on microarrays, alien sequences can serve as negative controls during the course of hybridization experimentation to assess the stringency (i.e., specificity) of target-to-probe hybridization. (2) Alien oligonucleotides spotted on micro-arrays, in combination with their complementary sequences used as in-spike controls can enable the experimenter to gauge the robustness of both the overall target labeling and hybridization efficiency. (3) When alien probe sequences are present within each sub-array on the biochip, they allow regional (intra-slide) effects of hybridization to be ascertained. (4) Alien oligonucleotides can also be used as in-spot controls and act as references so that inter-slide differences can be measured relative to a consistent control. (5) Detectably labeled alien sequences can be used to normalize the signal intensities of the samples under analysis on a per spot basis. Also, (6) in situ alien sequences may also be used to quality control the DNA microarray printing process.

In a first aspect, the present invention provides methods of identifying nucleotide sequences that are alien to a selected population.

Generating or Selecting Alien Sequences

As mentioned above, a nucleotide sequence is considered “alien” to a particular source or collection of nucleic acids if it does not hybridize with nucleic acids in the source or collection. For example, if the source or collection is mRNA or cDNA, then an oligonucleotide has a sequence that is “alien” to the mRNA or cDNA if its complement is not present in the mRNA or cDNA. Preferred alien oligonucleotides of the invention have complementary sequences that are maximally dissimilar from (i.e., non-identical to) those present in the source or collection.

When comparing polynucleotide sequences, two sequences are said to be “identical” if the sequence of nucleotides in the two sequences is the same when aligned for maximum correspondence. Comparisons between two sequences are typically performed by comparing the sequences over a comparison window to identify and compare local regions of sequence similarity. A “comparison window” refers to a segment of at least about 20 contiguous positions, usually 30 to about 75, or 40 to about 50, in which a sequence may be compared to a reference sequence of the same number of contiguous positions after the two sequences are optimally aligned.

Any of a wide variety of selection methods, systems or strategies that lead to the generation of oligonucleotides alien to a source or collection of nucleic acids can be used in the practice of the present invention. Such methods may, for example, be based on the use of an algorithm.

The present invention provides such an algorithm, in which the underlying logic is that of “partially reversing” the mathematical logic of the standard Hidden Markov Model. Such standard models are used to generate model sequences of DNA, RNA, proteins as well as other biological molecules, based on the statistics of known real (i.e., naturally occurring) sequences. Model sequences are generated based on sets of sequence symbol occurrences. For example, given the measured nearest neighbor frequencies (i.e., how often one nucleotide follows another) one then draws and outputs “randomly” from that set proportional to those frequencies. A very wide range of sequences statistics can be employed, from the simplest, the occurrence frequencies of the individual symbols, through all possible nearest neighbor frequencies to arbitrary spaced sequences frequencies.

A first approach used by the Applicants with the goal of generating “alien” or maximally dissimilar sequences from known real sequences was to perform a complete “reversal” of the statistics (i.e., to invert the sets of occurrence probability from most likely to least likely). However, when this strategy was tested over a very large set of sequences statistics, it did not work.

What did work in generating model sequences which are maximally dissimilar from those employed to obtain the sequence statistics, was to use a Markov process, in which, at an adjustable frequency, one draws from the measured real statistics but inversely proportional to those frequencies (or probability distributions). The sequence generated by this process contains, scattered throughout its length, intermittent highly improbable sequence patterns or subsequences. The frequency with which one switches between draws from the measured real sequence occurrence frequencies proportional to those frequencies and inversely proportional to those frequencies and inversely, ranges from one in five to one in ten. The selection of this ratio is partly a function of which sets of sequence statistics are used.

In the generation of maximally dissimilar DNA or mRNA complement sequences for microarray controls, preferably in the length range of 50 to 70 nucleotides, codon occurrence and codon boundary di-nucleotide frequencies were used for a range of inverse proportional inverse probability draws on these two statistics. This process was then followed by two filters, including: (1) a full genome sequence similarity search of all known or predicted protein coding regions, and (2) the calculation of TMs for all possible mRNA annealings for those with any sequence similarities above 60% identity and/or with matching runs longer than 18 nucleotides. All generated sequences with predicted annealing temperature above 37° C. or runs of twenty identities were eliminated. The TMs (i.e., midpoint disassociation temperatures) were calculated using multiple public domain software which included nucleotide stacking energies. This resulted in approximately one predicted “alien” or non-mRNA annealing oligo for every 5,000 genome coding regions in the higher animal and plant eukaryotic genomes currently known. Sets of these alien sequences were then synthesized and placed on “long oligo” microarray chips and physically tested for their annealing to real mRNA and/or cDNA samples. With rare exceptions (of one in ten), no detectable annealing was observed under standard experimental conditions for 70mer oligo array chips for 21,000 mouse genes. These alien sequences then define a set of negative controls.

A set of microarray “alien positive controls” was then generated from the above set of alien oligo negative control sequences using the following algorithm. First all possible set of three to five sequentially cancatinated alien oligos as defined above were generated in silico. These were investigated for the incidental creation of a sequence crossing the boundary between the cancatinated alien oligos that have a significant match or predicted annealing TM above 37° C. to any of the non-alien oligos on the micro-array targeted. Only those that showed no such matches or higher TMs were selected. These oligos were then physically synthesized as “positive alien gene” controls and tested for their ability to only anneal to their complementary alien oligos.

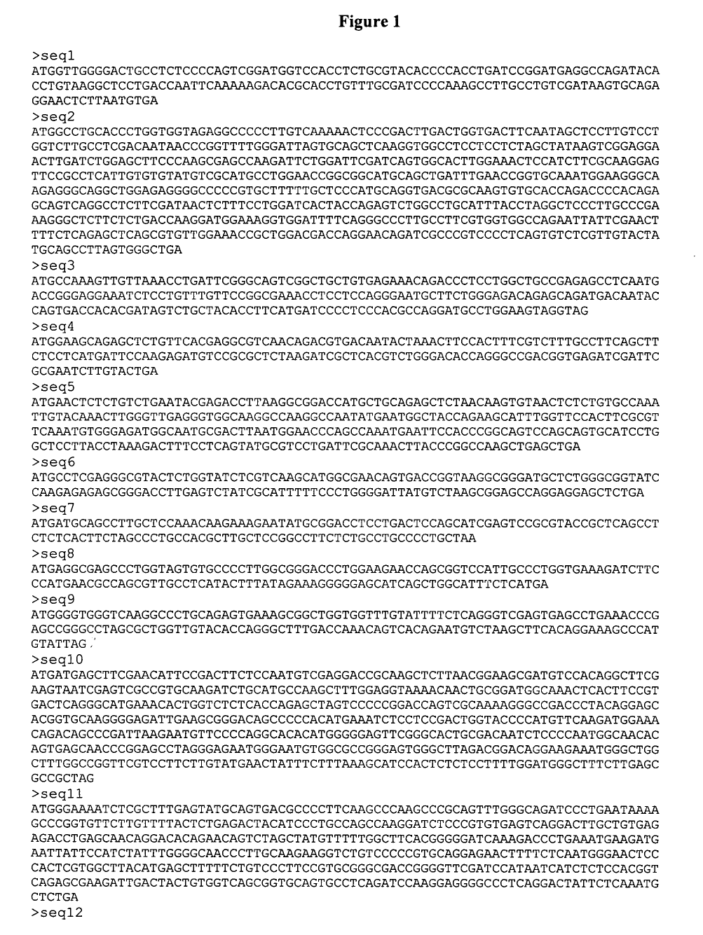

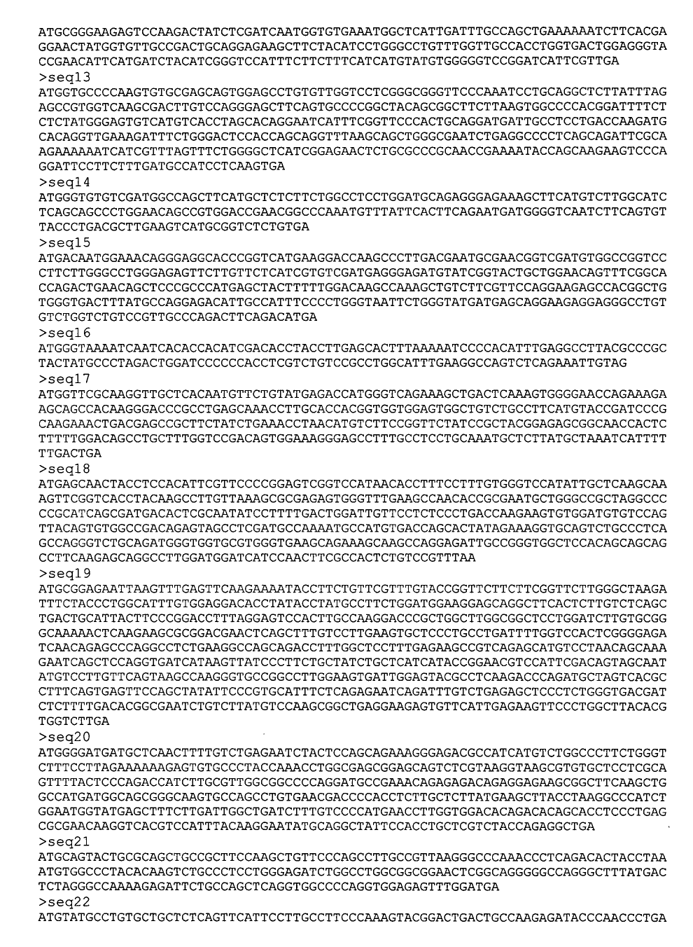

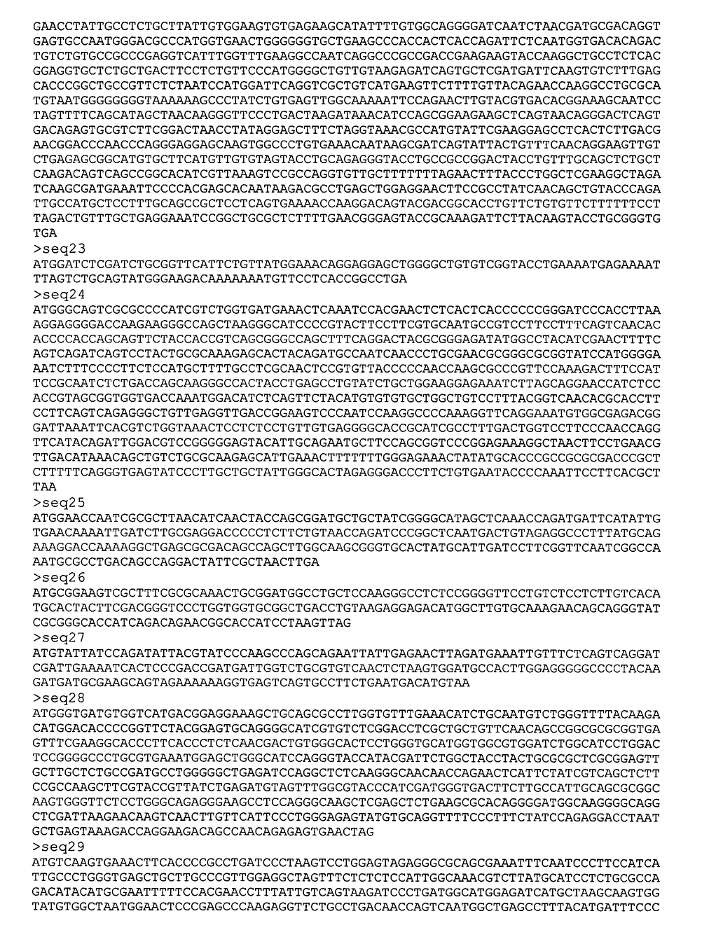

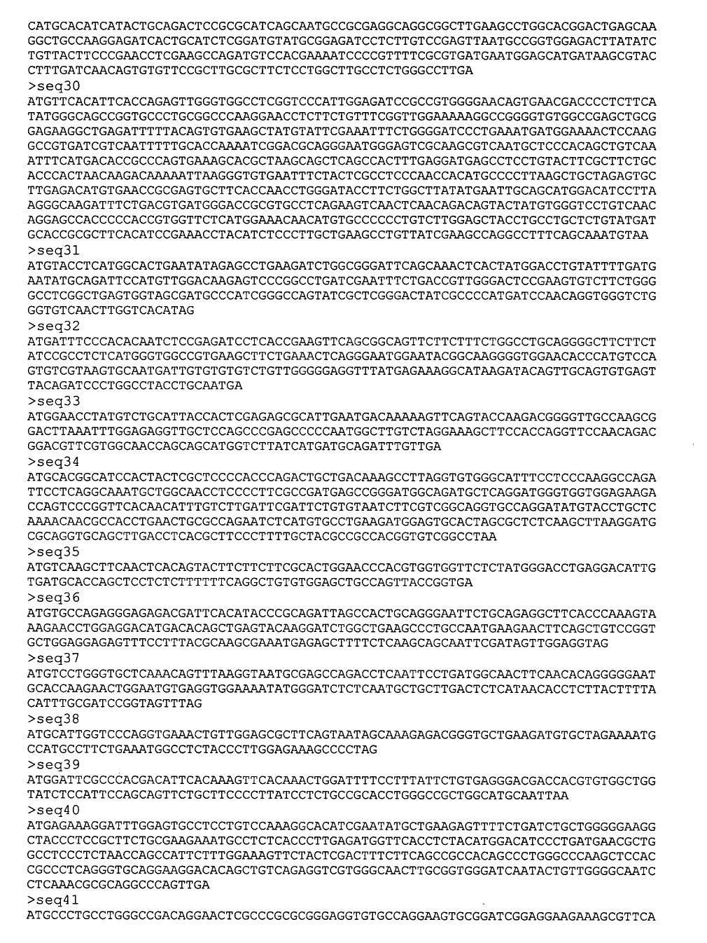

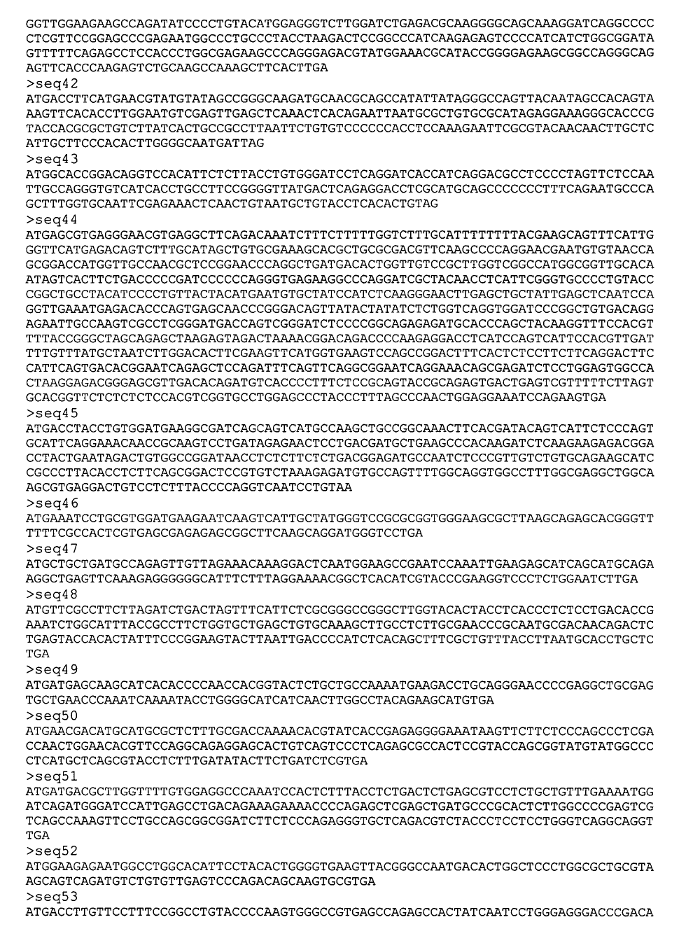

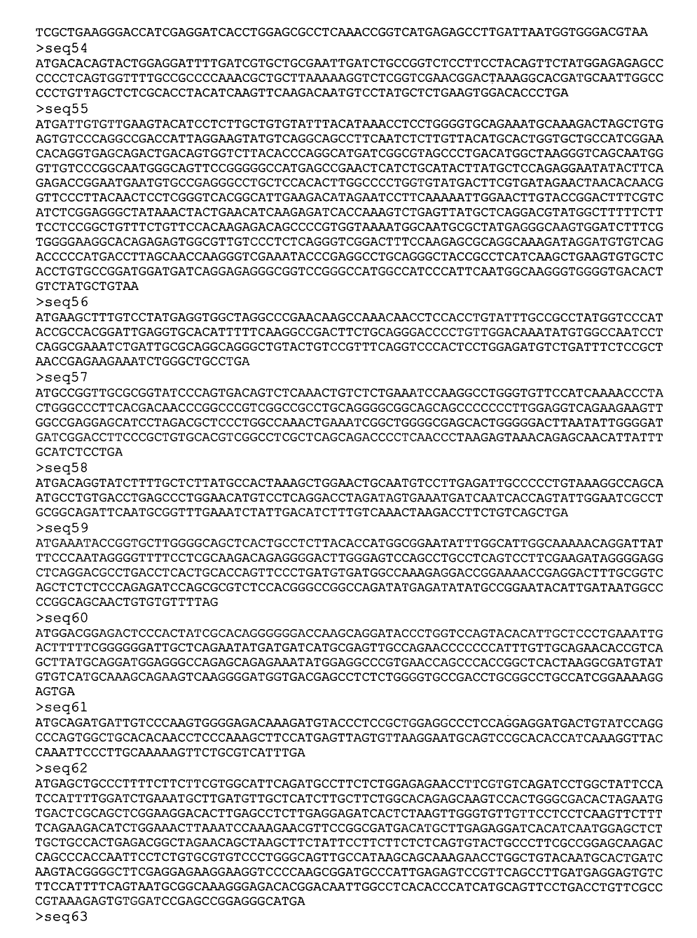

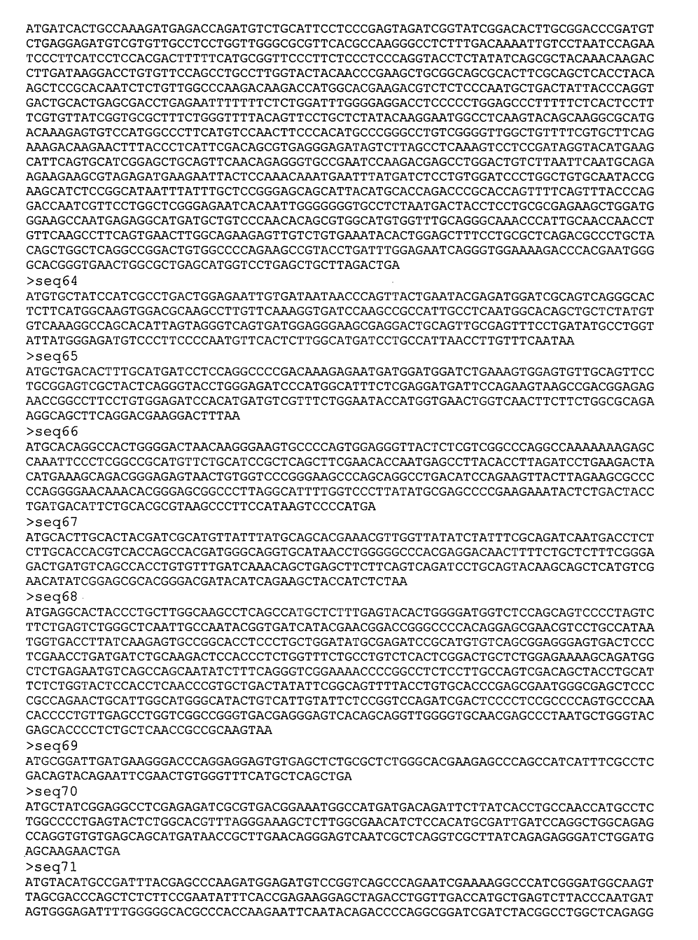

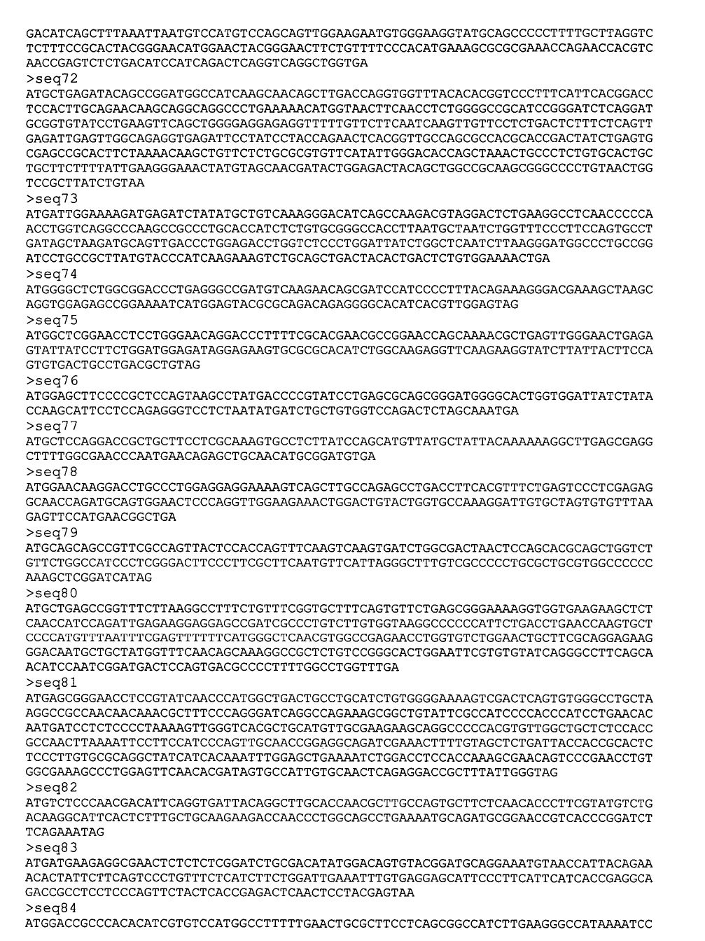

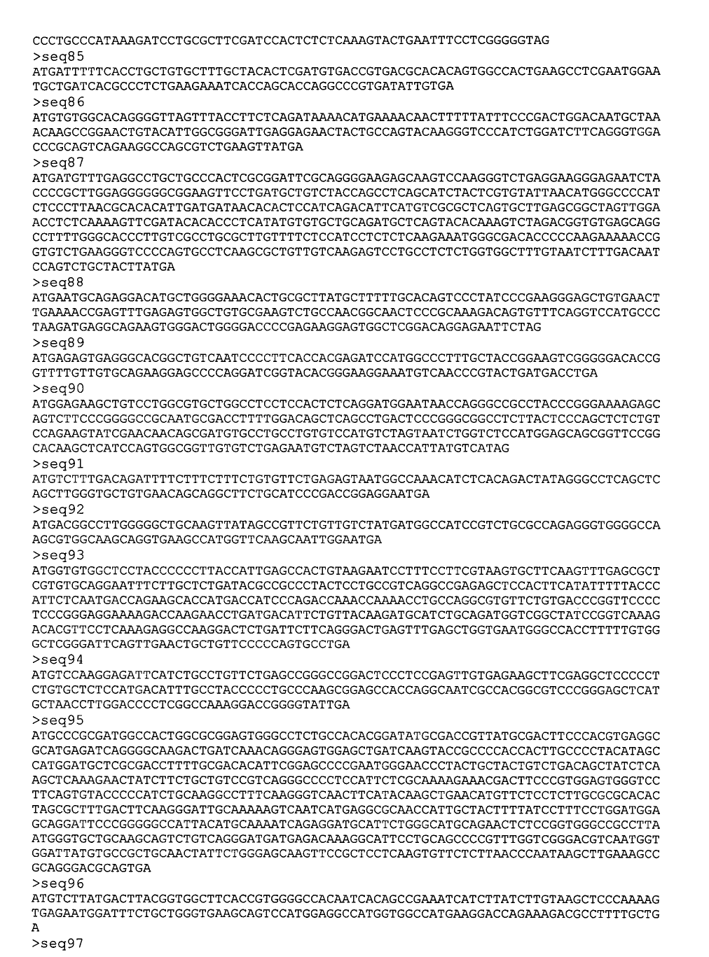

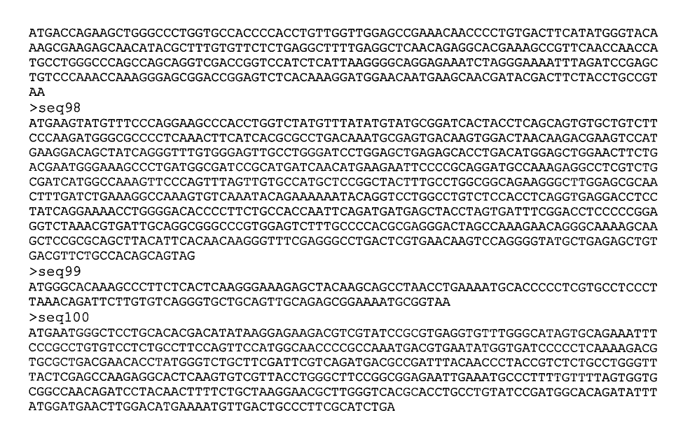

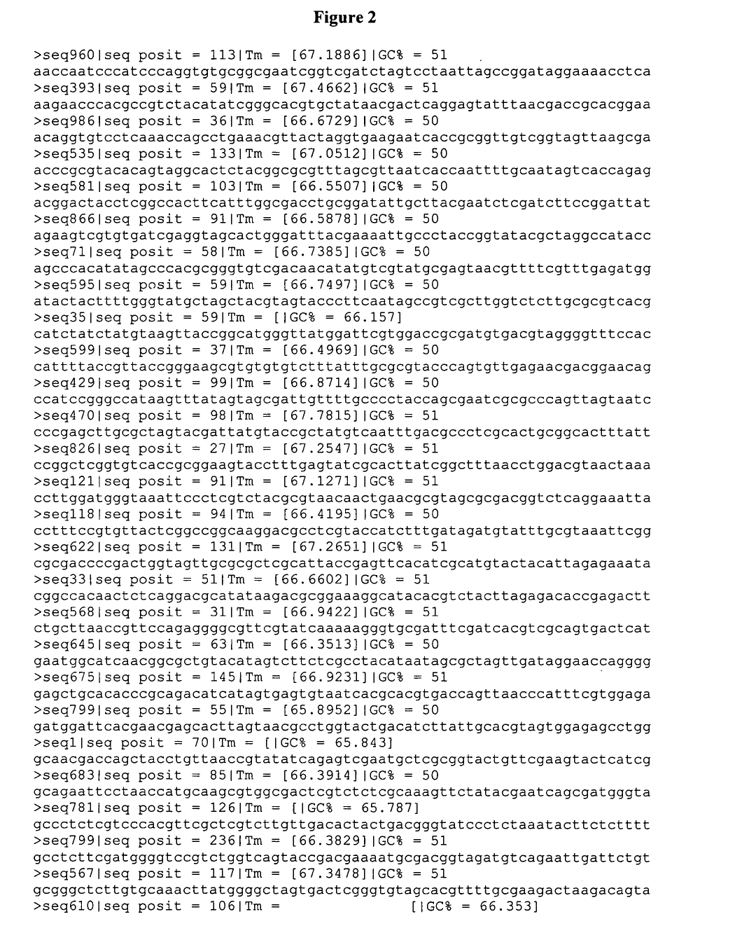

FIG. 1 shows about 100 sequences (of about 1000) that were generated using the inventive alien cDNA algorithm described above, by inverting sequences 35% of the time. FIG. 2 shows about 50 oligonucleotides identified as alien to mouse cDNA by the inventive algorithm and useful for hybridization applications.

In light of the inventive results described herein, those of ordinary skill in the art will appreciate that other algorithms may be employed or developed, for example, to include filter steps that, for example, verify the degree of “alien”ness of the selected sequence by comparing the generated oligonucleotide sequences to the organism's genome (if available) or cDNA by using any of a large number of sequence comparison programs.

A variety of methods for determining relationships between two or more sequences (e.g., identity, similarity and/or homology) are available, and well known in the art. The methods include manual alignment, computer assisted sequence alignment and combinations thereof. A number of algorithms (which are generally computer implemented) for performing sequence alignment are widely available, or can be produced by one of skill in the art. These methods include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.).

For example, a software for performing sequence identity (and sequence similarity) analysis using the BLAST algorithm is described in Altschul et al., J. Mol. Biol., 1990, 215: 403-410. This software is publicly available, e.g., through the National Center for Biotechnology Information on the World Wide Web at ncbi.nlm.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extensions of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP (BLAST Protein) program uses as defaults a wordlength (W) of 3, an expectation (E) of 10, and the BLOSUM62 scoring matrix (see, Henikoff& Henikoff, Proc. Natl. Acad. Sci. USA, 1989, 89:10915).

Additionally, the BLAST algorithm performs a statistical analysis of the similarity between two sequences (see, e.g., Karlin & Altschul, Proc. Natl. Acad. Sci. USA, 1993, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, or less than about 0.01, and or even less than about 0.001.

Another example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (J. Mol. Evol. 1987, 35: 351-360). The method used is similar to the method described by Higgins & Sharp (CABIOS, 1989, 5: 151-153). The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their nucleotide coordinates for regions of sequence comparison.

An additional example of an algorithm that is suitable for multiple DNA sequence alignments is the CLUSTALW program (J. D. Thompson et al., Nucl. Acids. Res. 1994, 22: 4673-4680). CLUSTALW performs multiple pairwise comparisons between groups of sequences and assembles them into a multiple alignment based on homology. Gap open and Gap extension penalties can be, e.g., 10 and 0.05 respectively.

An algorithm for the selection of alien sequences may also include filter steps that check for TM, % GC content, low-complexity regions and self hybridization. A large number of softwares (including those described above) are available and can be used to carry out these steps.

Alien Oligonucleotide Preparation

In another aspect, the present invention provides isolated oligonucleotides or nucleic acids that are alien to a given source or collection of nucleic acids. As will be appreciated by one skilled in the art, alien oligonucleotides may be of different lengths, depending on their intended use (as negative control, normalization and/or quantification tool or as in-spike control). For example, alien oligonucleotides may contain a single alien sequence. Alternatively, an alien oligonucleotide may contain at least two alien sequences linked to one another. Inventive oligonucleotides provided herein also include those polynucleotides that contain anti-alien sequences. For example, as described herein, it will often be desirable to prepare anti-alien sequences for use in hybridization reactions. In some embodiments, such sequences are prepared by polymerization directed by an alien gene.

Alien and anti-alien oligonucleotides of the invention may be prepared by any of a variety of chemical techniques well-known in the art, including, for example, chemical synthesis and polymerization based on a template (see, for example, S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; see also for example, U.S. Pat. No. 4,458,066).

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytrityl (or DMT) group at the 5′-position. After base base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on commercial oligo synthesizers such as the Perkin Elmer/Applied Biosystems Division DNA synthesizer. Such a synthesis is described in Example 2.

Oligonucleotides can also be custom made and ordered from a variety of commercial sources well-known in the art, including, for example, the Midland Certified Reagent Company (mcrc@oligos.com), The Great American Gene Company (available on the World Wide Web at genco.com), ExpressGen Inc. (available on the World Wide Web at expressgen.com), Operon Technologies Inc. (Alameda, Calif.) and many others.

Purification of oligonucleotides of the invention, where necessary, may be carried out by any of a variety of methods well-known in the art. Purification of oligonucleotides is typically performed by either by native acrylamide gel electrophoresis or by anion-exchange HPLC as described, for example, by Pearson and Regnier (J. Chrom. 1983, 255: 137-149). The sequence of the synthetic oligonucleotides can be verified using the chemical degradation method of Maxam and Gilbert (in Grossman and Moldave (Eds.), Academic Press, New York, Methods in Enzymology, 1980, 65: 499-560).

Assembling Arrays

The present invention provides nucleic acid arrays in which at least one spot contains an alien oligonucleotide. More specifically, inventive nucleic acids arrays comprise a solid support, and a plurality of nucleic acid probes attached to the solid support at discrete locations, wherein at least one the probes is an alien probe in that it has a sequence that is alien to a hybridizing mixture to be hybridized to the array.

Microarrays generally have sample spot sizes of less than 200 μm diameter, and generally contain thousands of spots per slide. For gene-expression analysis, each microarray preferably contain at least about 1,000, 5,000, 10,000, 50,000, 100,000, or 500,000 spots. The probes are printed (or attached) to the surface of the substrate, and the number of probes per unit area of the print surface is called the print density. The print surface corresponds to that area of the substrate on which the individual probes are printed, plus the surface area between the individual probes. If there are two or more groupings of a substantial number of probes on the substrate surface separated by surface area in which few or no probes are printed, the print surface includes the surface area between probes of a group but not the surface area of the substrate between groupings. For gene expression analysis, the print density is preferably high so that a large number of probes can fit on the substrate. Preferably, the print density is at least about 200, 500, 1,000, 5,000, 10,000, 20,000, or 40,000 probes per cm².

There are two standard types of DNA microarray technology in terms of the nature of the arrayed DNA sequence. In the first format, probe cDNA sequences (typically 500 to 5,000 bases long) are immobilized to a solid surface and exposed to a plurality of targets either separately or in a mixture. In the second format, oligonucleotides (typically 20-80-mer oligos) or peptide nucleic acid (PNA) probes are synthesized either in situ (i.e., directly on-chip) or by conventional synthesis followed by on-chip attachment, and then exposed to labeled samples of nucleic acids. In the present invention, microarrays of the second type are preferably used.

In the practice of the methods of the invention, investigators may either buy commercially available arrays (for example, from Affymetrix Inc. (Santa Clara, Calif.), Illumina, Inc. (San Diego, Calif.), Spectral Genomics, Inc. (Houston, Tex.), and Vysis Corporation (Downers Grove, Ill.)), or generate their own starting microarrays (i.e., arrays to which at least one alien oligonucleotide is to be spotted). Methods of making and using arrays are well known in the art (see, for example, S. Kern and G. M. Hampton, Biotechniques, 1997, 23:120-124; M. Schummer et al., Biotechniques, 1997, 23:1087-1092; S. Solinas-Toldo et al., Genes, Chromosomes & Cancer, 1997, 20: 399-407; M. Johnston, Curr. Biol. 1998, 8: R171-R174; D. D. Bowtell, Nature Gen. 1999, Supp. 21:25-32; D. J. Lockhart and E. A. Winzeler, Nature, 2000, 405: 827-836; M. Cuzin, Transfus. Clin. Biol. 2001, 8:291-296; M. Gabig and G. Wegrzyn, Acta Biochim. Pol. 2001, 48: 615-622; and V. G. Cheung et al., Nature, 2001, 40: 953-958).

Arrays comprise a plurality of probes immobilized to discrete spots (i.e., defined locations or assigned positions) on a substrate surface. Substrate surfaces for use in the present invention can be made of any of a variety of rigid, semi-rigid or flexible materials that allow direct or indirect attachment (i.e., immobilization) of probes (including alien oligonucleotides) to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may preferably be used (see, for example, U.S. Pat. No. 6,063,338).

The presence of reactive functional chemical groups (such as, for example, hydroxyl, carboxyl, amino groups and the like) on the material can be exploited to directly or indirectly attach probes including alien oligonucleotide sequences to the substrate surface. Methods of attachment (or immobilization) of oligonucleotides on substrate supports have been described and are well-known to those skilled in the art (see, for example, U. Maskos and E. M. Southern, Nucleic Acids Res. 1992, 20: 1679-1684; R. S. Matson et al., Anal. Biochem. 1995, 224; 110-116; R. J. Lipshutz et al., Nat. Genet. 1999, 21: 20-24; Y. H. Rogers et al., Anal. Biochem. 1999, 266: 23-30; M. A. Podyminogin et al., Nucleic Acids Res. 2001, 29: 5090-5098; Y. Belosludtsev et al., Anal. Biochem. 2001, 292: 250-256).

Methods of preparation of oligonucleotide-based arrays that can be used to attach probes to surface support of microrrays include: synthesis in situ using a combination of photolitography and oligonucleotide chemistry (see, for example, A. C. Pease et al., Proc. Natl. Acad. Sci. USA 1994, 91: 5022-5026; D. J. Lockhart et al, Nature Biotech. 1996, 14: 1675-1680; S. Singh-Gasson et al., Nat. Biotechn. 1999, 17: 974-978; M. C. Pirrung et al., Org. Lett. 2001, 3: 1105-1108; G. H. McGall et al., Methods Mol. Biol. 2001, 170; 71-101; A. D. Barone et al., Nucleosides Nucleotides Nucleic Acids, 2001, 20: 525-531; J. H. Butler et al, J. Am. Chem. Soc. 2001, 123: 8887-8894; E. F. Nuwaysir et al., Genome Res. 2002, 12: 1749-1755). The chemistry for light-directed oligonucleotide synthesis using photo labile protected 2′-deoxynucleoside phosphoramides has been developed by Affymetrix Inc. (Santa Clara, Calif.) and is well known in the art (see, for example, U.S. Pat. Nos. 5,424,186 and 6,582,908).

Alternatively or additionally, oligo probes may first be prepared or print-ready oligonucleotide (e.g., 60-70 mers) sets that are commercially available for human, mouse and other organism (see, for example, http://www.cgen.com, http://www.operon.com) may be obtained and then attached to the array surface. Similarly, alien oligonucleotides are first synthesized and then immobilized on the surface of a microarray.

In these cases, the preparation of microarrays is preferably carried out by high-speed printing robotics. The established robotic spotting technique (U.S. Pat. No. 5,807,522) uses a specially designed mechanical robot, which produces a probe spot on the microarray by dipping a pin head into a fluid containing an off-line synthesized nucleic acid molecule and then spotting it onto the slide at a pre-determined position. Washing and drying of the pins are required prior to the spotting of a different probe in the microarray. In current designs of such robotic systems, the spotting pin, and/or the stage carrying the microarray substrates move along the XYZ axes in coordination to deposit samples at controlled positions of the substrates.

In addition to the established quill-pin spotting technologies, there are a number of microarray fabrication techniques that are being developed. These include the inkjet technology and capillary spotting.

Example 2 describes the printing of alien oligonucleotides to the surface of oligo slides (CodeLink, Amersham Biosciences, Piscataway, N.J.), which also contain human and mouse positive control spots.

As mentioned above, microarrays provided by the present invention are arrays containing a plurality of oligo probes and in which at least one spot contains an alien oligonucleotide. In certain preferred embodiments, an alien oligonucleotide is printed at more than one spot on the array. For example, an inventive microarray may contain, in addition to a plurality of oligo probes, a representative collection of spots containing the same or different concentrations of the alien oligonucleotide. Alternatively, all the spots on an inventive microarray may contain the same or different concentrations of the alien oligonucleotide.

In other embodiments, an inventive microarray contains at least two different alien oligonucleotides. These alien oligonucleotides may be spotted randomly throughout the whole array or they may be present in specific areas of the substrate surface, for example, forming probe elements (i.e., sub-arrays) containing only one type of alien oligonucleotide.

In still other embodiments, an inventive microarray contains alien oligonucleotides of different sizes. For example, an inventive microarray may contain a first oligonucleotide comprising a single alien sequence and a second oligonucleotide comprising at least two different alien sequences. The presence of both types of alien oligonucleotides on the microarray may, for example, allow two different types of controls to be performed.

The present invention also provides sets of microarrays that all contain identical probe elements (i.e., defined sets of spots) except for one microarray (or part of one microarray), which contains no alien oligonucleotide and another microarray (or part of a microarray) that contains the same probe elements but with fixed amount(s) of alien oligonucleotide.

Labeling of Nucleic Acid Molecules

In certain embodiments, nucleic acid molecules of the hybridzing mixture are labeled with a detectable agent before hybridization. In other embodiments, complementary sequences of alien oligonucleotides (i.e., anti-alien oligonucleotides), which are added to the hybridization sample before hybridization, are also labeled. In both cases, the role of a detectable agent is to facilitate detection and to allow visualization of hybridized nucleic acids. Preferably, the detectable agent is selected such that it generates a signal which can be measured and whose intensity is related to the amount of labeled nucleic acids present in the sample being analyzed. The detectable agent is also preferably selected such that it generates a localized signal, thereby allowing spatial resolution of the signal from each spot on the array.

The association between the nucleic acid molecule and detectable agent can be covalent or non-covalent. Labeled nucleic acids can be prepared by incorporation of or conjugation to a detectable moiety. Labels can be attached directly to the nucleic acid or indirectly through a linker. Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Many methods for labeling nucleic acid molecules are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes or of enzymes; chemical modifications of nucleic acids making them detectable immunochemically or by other affinity reactions; and enzyme-mediated labeling methods, such as random priming, nick translation, PCR and tailing with terminal transferase. More recently developed nucleic acid labeling systems include, but are not limited to: ULS (Universal Linkage System; see, for example, R. J. Heetebrij et al., Cytogenet. Cell. Genet. 1999, 87: 47-52), photoreactive azido derivatives (see, for example, C. Neves et al., Bioconjugate Chem. 2000, 11: 51-55), and alkylating agents (see, for example, M. G. Sebestyen et al., Nat. Biotechnol. 1998, 16: 568-576).

Any of a wide variety of detectable agents can be used in the practice of the present invention. Suitable detectable agents include, but are not limited to: various ligands, radionuclides (such as, for example, ³²P, ³⁵S, ³H, ¹⁴C, ¹²⁵I, ¹³¹I and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, e.g., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

In certain preferred embodiments, nucleic acid molecules (or anti-alien oligonucleotides) are fluorescently labeled. Numerous known fluorescent labeling moieties of a wide variety of chemical structures and physical characteristics are suitable for use in the practice of this invention. Suitable fluorescent dyes include, but are not limited to: Cy-3™, Cy 5™, Texas red, FITC, Alexa-488, phycoerythrin, rhodamine, fluorescein, fluorescein isothiocyanine, carbocyanine, merocyanine, styryl dye, oxonol dye, BODIPY dye (i.e., boron dipyrromethene difluoride fluorophore), and equivalents, analogues, derivatives or combinations of these molecules. Similarly, methods and materials are known for linking or incorporating fluorescent dyes to biomolecules such as nucleic acids (see, for example, R. P. Haugland, “Molecular Probes: Handbook of Fluorescent Probes and Research Chemicals 1992-1994”, 5^{th} Ed., 1994, Molecular Probes, Inc.). Fluorescent labeling dyes as well as labeling kits are commercially available from, for example, Amersham Biosciences, Inc. (Piscataway, N.J.), Molecular Probes, Inc. (Eugene, Oreg.), and New England Biolabs, Inc. (Berverly, Mass.).

Favorable properties of fluorescent labeling agents to be used in the practice of the invention include high molar absorption coefficient, high fluorescence quantum yield, and photostability. Preferred labeling fluorophores exhibit absorption and emission wavelengths in the visible (i.e., between 400 and 750 nm) rather than in the ultraviolet range of the spectrum (i.e., lower than 400 nm).

Hybridization products may also be detected using one of the many variations of the biotin-avidin technique system, which that are well known in the art. Biotin labeling kits are commercially available, for example, from Roche Applied Science (Indianapolis, Ind.) and Perkin Elmer (Boston, Mass.).

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and allowing emission of a fluorescence signal. The use of molecular beacons as detectable moieties is well-known in the art (see, for example, D. L. Sokol et al., Proc. Natl. Acad. Sci. USA, 1998, 95: 11538-11543; and U.S. Pat. Nos. 6,277,581 and 6,235,504). Aptamer beacons are similar to molecular beacons except that they can adopt two or more conformations (see, for example, O. K. Kaboev et al., Nucleic Acids Res. 2000, 28: E94; R. Yamamoto et al., Genes Cells, 2000, 5: 389-396; N. Hamaguchi et al., Anal. Biochem. 2001, 294: 126-131; S. K. Poddar and C. T. Le, Mol. Cell. Probes, 2001, 15: 161-167).

Multiple independent or interacting labels can also be incorporated into the nucleic acids. For example, both a fluorophore and a moiety that in proximity thereto acts to quench fluorescence can be included to report specific hybridization through release of fluorescence quenching (see, Tyagi et al., Nature Biotechnol. 1996, 14: 303-308; Tyagi et al., Nature Biotechnol. 1998, 16: 49-53; Kostrikis et al., Science, 1998, 279: 1228-1229; Marras et al., Genet. Anal. 1999, 14: 151-156; U.S. Pat. Nos. 5,846,726, and 5,925,517)

A “tail” of normal or modified nucleotides may also be added to nucleic acids for detectability purposes. A second hybridization with nucleic acid complementary to the tail and containing a detectable label (such as, for example, a fluorophore, an enzyme or bases that have been radioactively labeled) allows visualization of the nucleic acid molecules bound to the array (see, for example, system commercially available from Enzo Biochem Inc., New York, N.Y.).

The selection of a particular nucleic acid labeling technique will depend on the situation and will be governed by several factors, such as the ease and cost of the labeling method, the quality of sample labeling desired, the effects of the detectable moiety on the hybridization reaction (e.g., on the rate and/or efficiency of the hybridization process), the nature of the detection system to be used, the nature and intensity of the signal generated by the detectable label, and the like.

Hybridization

According to the methods provided, an inventive nucleic acid array (i.e., a microarray in which at least one spot contains an alien oligonucleotide) is contacted with a hybridizing mixture comprising a plurality of nucleic acids under conditions wherein the nucleic acids in the mixture hybridize to the probes on the array.

The hybridization reaction and washing step(s), if any, may be carried out under any of a variety of experimental conditions. Numerous hybridization and wash protocols have been described and are well-known in the art (see, for example, J. Sambrook et al., “Molecular Cloning: A Laboratory Manual”, 1989, 2^{nd} Ed., Cold Spring Harbour Laboratory Press: New York; P. Tijssen “Hybridization with Nucleic Acid Probes—Laboratory Techniques in Biochemistry and Molecular Biology (Part II)”, Elsevier Science, 1993; and “Nucleic Acid Hybridization”, M. L. M. Anderson (Ed.), 1999, Springer Verlag: New York, N.Y.).

The hybridization and/or wash conditions may be adjusted by varying different factors such as the hybridization reaction time, the time of the washing step(s), the temperature of the hybridization reaction and/or of the washing process, the components of the hybridization and/or wash buffers, the concentrations of these components as well as the pH and ionic strength of the hybridization and/or wash buffers.

In certain cases, the specificity of hybridization may further be enhanced by inhibiting or removing repetitive sequences. By excluding repetitive sequences from the hybridization reaction or by suppressing their hybridization capacity, one prevents the signal from hybridized nucleic acids to be dominated by the signal originating from these repetitive-type sequences (which are statistically more likely to undergo hybridization).

Removing repetitive sequences from a mixture or disabling their hybridization capacity can be accomplished using any of a variety of methods well-known to those skilled in the art. Preferably, the hybridization capacity of highly repeated sequences is competitively inhibited by including, in the hybridization mixture, unlabeled blocking nucleic acids.

Microarray-based hybridization reactions in which alien oligonucleotides may serve as controls include a large variety of processes. For example, they may be useful in gene expression methods, such as those developed and used in pharmacogenomic research (see, for example, M. Srivastava et al., Mol. Med. 1999, 5: 753-767; and P. E. Blower et al., Pharmacogen. J. 2002, 2: 259-271); in drug discovery (see, for example, C. Debouk and P. N. Goodfellow, Nat. Genet. 1999, 21: 48-50; and A. Butte, Nat. Rev. Drug Discov. 2002, 1: 951-960), or in medicine and clinical research, for example, in cancer research (see, for example, J. DeRisi et al., Nat. Genet. 1996, 14: 457-460; C. S. Cooper, Breast Cancer Res. 2001, 3: 158-175; S. B. Hunter and C. S. Moreno, Front Biosci. 2002, 7: c74-c82; R. Todd and D. T. Wong, J. Dent. Res. 2002, 81: 89-97).

In another aspect, the inventive provides methods of using alien oligonucleotides and their complements in microarray-based hybridization experiments for different control purposes.

Alien Sequences as Negative Controls

In certain embodiments of the invention, alien oligonucleotide sequences are used to serve as a negative control during the course of the microarray experimentation. Negative controls are valuable when assessing the stringency of target-to-probe hybridization. For example, the selectivity of hybridization is known to be paramount to the accurate reflection of differential gene expression.

When present on a microarray, inventive alien oligonucleotides (i.e., molecules comprising sequences selected for their inability to hybridize nucleic acids of the source or collection under analysis) can act as negative controls. If a detectable signal can be measured from spots containing alien sequences, then hybridization conditions are not stringent and lead to significant cross-hybridization reactions, which, in turn, adversely affect the measured differential gene expression.

Use of Alien Sequences to Quantify Hybridization Sample Components

The present invention also provides methods that allow quantification of hybridizing sample components. Such methods are based on the use of microarrays containing alien oligonucleotides and on the addition of their complements (i.e., anti-alien sequences) to the hybridizing mixture before hybridization.

More specifically, inventive methods comprise providing a hybridizing mixture comprising a plurality of nucleic acids; and hybridizing the hybridizing mixture to a nucleic acid array of the invention, wherein the step of providing a hybridizing mixture comprises providing a mixture containing at least one anti-alien hybridizing nucleic acid whose sequence comprises a sequence complementary to the alien probe present on the inventive nucleic acid array.

In certain preferred embodiments, a known amount of an anti-alien oligonucleotide is added to a sample containing at least one experimental hybridizing nucleic acid of unknown quantity, and the mixture thus obtained is processed and prepared for hybridization to a microarray containing the alien oligonucleotide. The processing and preparation include labeling of both the anti-alien sequence and test nucleic acids with the same detectable agent. The degree of anti-alien/alien hybridization may be relied upon to establish the amount of test sequences present in the hybridizing sample based on the relative extent of their hybridization to complementary oligo probes present on the microarray.

In preferred embodiments, the degree of hybridization between the anti-alien and alien oligonucleotides and/or between the hybridizing nucleic acid and oligonucleotide probe present on the array is determined by measuring the signal intensities from the detectable label attached to the hybridized targets.

More specifically, if, for example, the target nucleic acids have been fluorescently labeled, the amount of a particular sequence in the hybridizing mixture is determined by comparing the intensity of the fluorescence signal measured for the hybridized sequence to the intensity of the fluorescence signal measured for the anti-alien sequence hybridized to the alien oligonucleotide present on the microarray.

In other preferred embodiments, an unknown amount of the anti-alien oligonucleotide is added to a nucleic acid sample to be analyzed and the resulting mixture is processed as above, before hybridization to a microarray containing a known amount of the alien oligonucleotide. The quantification of hybridization sample components may then be carried out as described above.

In other preferred embodiments of the invention, different amounts of multiple alien/anti-alien pairs are used for comparative quantification of nucleic acids of the test sample. Using amounts of multiple alien/anti-alien pairs, that vary from rare, to low, to abundant and highly abundant provides reference signal intensities for widely different ranges of target amounts (or concentrations), and therefore can help improve the accuracy of the quantification of test sequences. Such a method may be particularly useful when the signal intensity vs. detectable label amount (which is equivalent to hybridized target amount) exhibits a deviation from linearity in one or more concentration ranges.

Use of Alien Sequences for Normalization

Also provided by the present invention are methods wherein alien oligonucleotides are used as controls for in situ normalization.

At present, differential gene expression relies on changes in the relative abundance of any given mRNA between a test and reference total RNA sample. Usually ratios are derived that identify if a test sample mRNA is up- or down-regulated with respect to a reference sample, however in many instances no appropriate reference sample exists. Such a problem is typically encountered when samples are collected over extended periods of time (i.e., clinical studies) and need to be compared to a common reference or in diseased patients where no applicable reference is available.

In certain preferred embodiments, a microarray has spots containing a mixture of known amounts of the alien oligonucleotide and of a probe able to detect target (or hybridizing) sequences. Such an arrangement allow in situ comparisons. This approach also provides a consistent standard (the fixed amount of alien oligonucleotide) that can be relied upon to allow inter-slide comparisons and inter-experiment comparisons even when experiments are carried out with rare samples, or over a long time spans.

In these particular instances, an alien sequence can be used as an in-spot control and act as the reference so that inter-slide expression differences can be measured relative to a consistent control.

For instance, if every spot in an array has a defined mixture of experimental probes to alien probes, the presence of the alien oligonucleotides allows the researcher to control for variations between and among spots (e.g., by hybridizing the array with a sample containing anti-alien sequences that are differently labeled from the target sequences.

Those of ordinary skill in the art will appreciate that it is not essential that every spot on the array contain alien oligonucleotide, though it will typically be desirable that the alien oligo be present in a representative collection of spots, for example, so that the researcher can have reasonable confidence in the general uniformity of the spots. It will also be appreciated that, although convenient, it is not essential that every spot containing the alien sequence contain the same ratio of alien and experimental probes; so long as the ratio for each spot is defined and known.

In these methods, normalization is performed according to standard techniques.

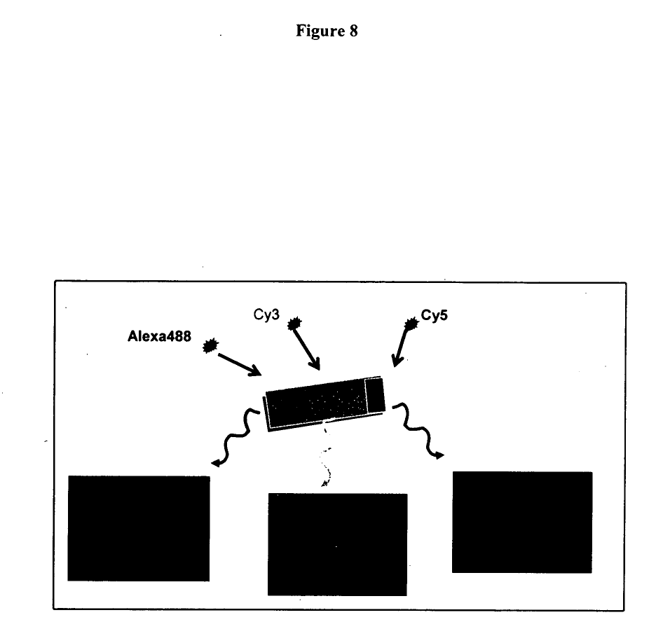

As shown on the scheme presented in FIG. 8, an alien 70mer probe can be co-printed with a gene specific probe on the microarray so that the two independent hybridizations can be measured within the same spot. A complimentary alien oligonucleotide labeled with a fluorescent dye can be employed to serve as the reference, and can be simply mixed with the labeled target at known concentration prior to hybridization. The test RNA signal intensity is then compared to the alien control allowing like inter-slide comparisons to be made across a large data sets.

Controlling Hybridization Sample Processing and Hybridization with Alien Sequences

Furthermore, when an alien oligonucleotide is present on an array, its complement may be added to the hybridizing sample, and processed (i.e., subjected to different treatments including labeling) together with the sample, and hybridized to an inventive microarray as a control for the processing/hybridizaton steps. If the alien oligonucleotide is present in spots at different locations on the chip, this strategy can also control intra-chip hybridization variation.

To give but one example, as described in the Examples, the present inventors have designed alien sequences that consist of four alien sequences that have been concatemerized behind a T7 promoter and to maintain polyadenylated tails. Upon transcription of the alien genes with T7 RNA polymerase, an alien transcript can be added to the total RNA input and act as an internal control during the course of cDNA generation, labeling, and hybridization. When alien probes, complementary to the alien gene, are included on the microarray, the experimenter can measure the extent of hybridization between the alien probe and the anti-alien nucleic acid in the labeled cDNA milieu to ascertain the overall labeling and hybridization efficiency. While this control does not definitively identify whether the labeling or hybridization may be at fault when there is a failure to detect fluorescent signal, it does allow the experimenter to identify if a problem has occurred and to compare the relative labeling efficiencies from experiment to experiment. One would anticipate that when the labeling and hybridization are successful, the relative signal intensity from the alien probe would be similar between slides. Similarly, regional effects of hybridization can be ascertained by including alien probe sequences within each sub-array on the chip. This comparative metric for inter-slide and intra-slide comparison is beneficial for quality control purposes.

Controlling for Array Manufacture using Alien Sequences

In another aspect, the invention provides methods that allow control of array manufacture. More specifically, when an alien oligonucleotide is present on an array, a standardized (i.e., a known amount, optionally labeled) complementary nucleic acid may be added to the hybridizing sample, and the extent of its hybridization to the alien sequence on the microarray can be used to assess the quantity of the array manufacture (e.g., the extent to which oligonucleotides were effectively coupled to the surface, etc).

Thus, according to the present invention, it is possible to analyze printed microarrays (e.g., prior to their experimental use, for example to ascertain if any spots are missing (and if so which ones), as well as to judge overall spot morphology and slide quality.

### EXEMPLIFICATION

The following examples describe modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Identification of Alien Sequences

The present invention provides systems for identifying “alien” sequences that are not found in the relevant population of nucleic acids being hybridized to an array. For instance, the invention provides systems for identifying sequences that are not present in the cDNA of a selected organism.

In particular, a software program was developed that allows the user to generate “alien” cDNA's for a particular organism. The program, the algorithm of which was described above, takes in a list of all known cDNA sequences for that particular organism (e.g., mouse). From this list, the program calculates the codon frequency of the sequences as well as dinucleotide or transition sequences at the codon boundary. These files can be stored and are specific for the organism from which the frequencies are generated. The program then generates cDNA (with start and stop codons) using the above frequencies. A small percentage of the time (as may be specified by the user), the generated frequencies are flipped such that the least frequent codon is now generated in the middle of the sequence. Such a sequence should be different from any cDNA occurring in the genome. The degree of “alien”ness of the sequence can be verified by comparing the generated sequences to the organism's genome (if available) or cDNA by using BLAST or another sequence comparison program. Oligos are then generated from the sequences by using another software program which checks for Tm and % GC content. The generated oligos are also compared to the organism genome or cDNA to verify that they do not hybridize to any part of the genome.

For example, FIG. 1 shows about 100 sequences (of about 1000) that were generated using the inventive alien cDNA software, by inverting sequences 35% of the time.

FIG. 2 shows about 50 sequences that were identified as alien to mouse cDNA and desirable for use in hybridization applications. The sequences were passed through oligo selection software to check Tm, % GC content, low-complexity regions and self hybridization. The software also checks by using two programs, Fuzznuc (EBI tool) and BLAST, whether the sequences have any similarity to cDNA from the organism in question. The oligos are then filtered by comparing them using BLAST against the organism's genome if available.

### Example 2

### Attaching Alien Sequences to Chips

Synthesis of alien oligonucleotides. Each of the 47 70mer alien oligonucleotide probes depicted in FIG. 2 was synthesized using an Expedite DNA synthetizer (Applied Biosystems, Framingham, Mass.) following standard protocols of phosphoramidite chemistry at a 200 mmol scale (S. L. Beaucage and R. P Iyer, Tetrahedron, 1992, 48: 2223-2311; S. L. Beaucage and R. P. Iyer, Tetrahedron, 1993, 49: 6123-6194). All alien oligonucleotides were modified at the 5′ terminus with a TFA-amino-C-6-phosphoramidite (Prime Organics, Lowell, Mass.) to enable subsequent covalent attachment of the oligonucleotide to a CodeLink (Amersham Biosciences) slide surface. After synthesis, oligonucleotides were cleaved and deprotected from the CPG support with concentrated ammonium hydroxide at 80° C. for 16 hours and lyophilized. The oligonucleotides were re-dissolved in 300 μL of water and then desalted on Performa SR DNA synthesis cleanup plates (EdgeBiosystems, Gaithersburg, Md.). All oligonucleotides were quality assessed by capillary electrophoresis (CombiSep, Ames, IA) and quantified by UV spectroscopic measurement.

Preparation of oligo slide. Alien oligonucleotides were then printed and linked to the surface of oligos slides (CodeLink, Amersham Biosciences, Piscataway, N.J.), which also contained human and mouse positive control spots. All the plates were prepared following the same protocol.

Alien oligonucleotides were arrayed in Greiner 384-well flat-bottom plates (600 pmol of alien oligonucleotide per well). After resuspension in water to 20 μM, the oligonucleotides (5 μL) were re-arrayed into 384-well, Genetix polystyrene V-bottom plates, which were then allowed to dry in a chemical hood. Before printing, 5 μL of 1× Printing Buffer (150 mM sodium phosphate, 0.0005% Sarcosyl) were added to each well. The plates were incubated at 37° C. for 30 minutes to aid resuspension of DNA, vigorously shaken on a flat-bed shaker for 1 minute, and centrifuged at 2000 rpm for 3 minutes. These plates were then placed into an OmniGrid® 100 microarrayer (GeneMachines, San Carlos, Calif.) for the preparation of oligos slides.

After completion of each print run, the slides were removed from the microarrayer and placed overnight in a sealed humidification chamber containing a saturated brine solution and lined with moist paper towels. The slides were then transferred to a slide rack (25 slides per rack), which was placed into a container filled with Pre-warmed Blocking Solution (50 mM 2-aminoethanol; 0.1 M Tris pH 9, 0.1% N-Lauroyl sarcosine) to completely cover the slides, and then shaken for 15 minutes. The slides were rinsed twice with de-ionised water by transferring the slide rack to water filled containers. The slide rack was then transferred to another container filled with pre-warmed Washing Solution (4×SSC, 0.1% N-Lauroyl sarcosine) to completely cover the slides, and then shaken for 30 minutes. After the slides were rinsed twice with de-ionized water, they were dried by centrifugation at 800 rpm for 5 minutes, and stored in a dessicator.

Terminal Deoxynucleotidyl Transferase Quality Control. A first set of slides were treated with Terminal Deoxynucleotidyl Transferase in the presence of dCTP-Cy3, so that all oligonucleotides attached to the slide could be visualized and their attachment assessed. The labeling was performed by adding 10 μL of 5× reaction buffer (containing 500 mM sodium cacodylate, pH 7.2, 1 mM 2-mercaptoethanol, and 10 mM COCl₂), 0.5 μL of Cy3-dCTP (Amersham), 2 μL of Terminal Deoxynucleotidyl Transferase (Amersham, 12 units/mL) and water to a final volume of 124 μL. The reaction solution was briefly vortexed and spinned. The slides were boiled for 10 minutes in ddH₂O and dried with a gentle air stream. The Terminal Transferase hybridization procedure, which was performed using a GeneTac Hybridization station (BST Scientific, Singapore), included an incubation cycle carried out at 37° C. for 2 hours followed by three washing steps.

After the slides were rinsed with 0.06×SSC, and then dried by centrifugation, they were scanned within the next 24 hours using an Axon GenePix 4000B scanner (Axon Instruments, Union City, Calif.). The resulting images were analyzed using the GenePix 3.0 software package.

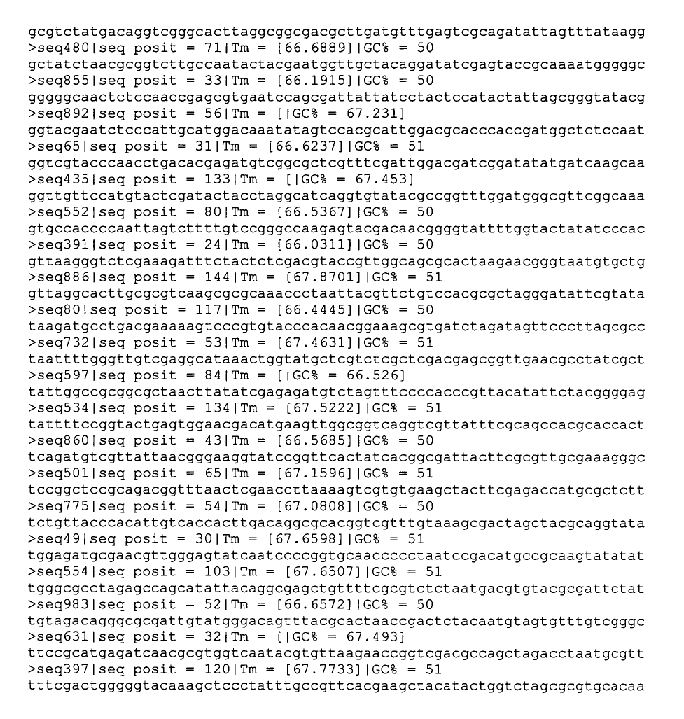

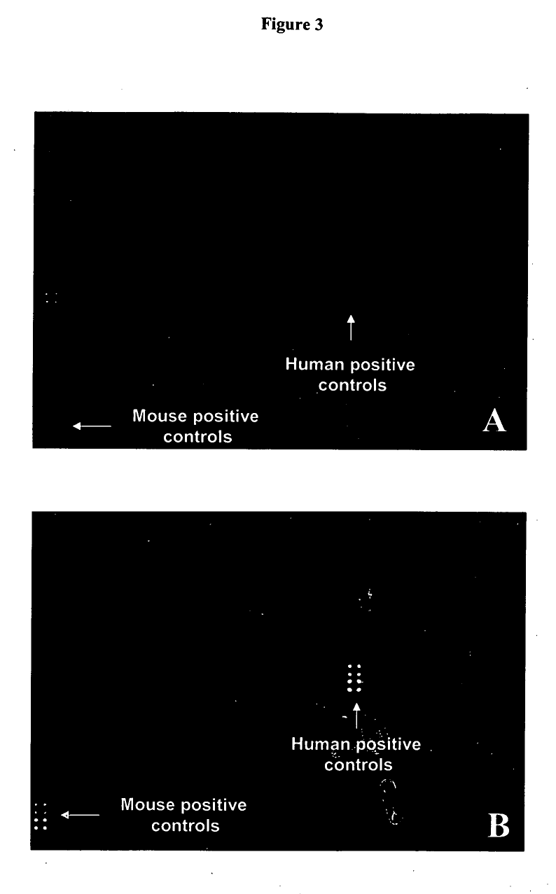

As shown in FIG. 3A, the labeled alien oligonucleotides attached to slides having undergone such a Terminal Deoxynucleotididyl Transferase process were readily detectable, as were the human and mouse positive controls.

A second set of slides was not treated with terminal deoxynucleotidyl transferase, and instead was hybridized with labeled mRNA from human (Stratagene's Universal RNA Human) and mouse (Stratagene's Universal RNA Mouse).

Labeling of Universal Mouse/Human RNA. Before hybridization, samples of both types of mRNA were labeled using the standard indirect labeling method developed by J. B. Randolph and A. S. Waggoner (Nucleic Acids Res. 1997, 25: 2923-2929). Human mRNA was labeled with Cy5™ and mouse mRNA was labeled with Cy3™. Briefly, aminoallyl dUTP was incorporated during the reverse transcription of the total RNAs. This modified cDNA in turn was labeled via a coupling between an N-hydroxysuccinimide activated ester of a fluorescent dye (Monoreactive Cy3 and Cy5 from Amersham) and the aminoallyl moiety of the dUTP, following a modified version of the Atlas Powerscript Fluorescent Labeling Kit (BD Biosciences Clontech, Palo Alto, Calif.) protocol.

Hybridization to alien oligonucleotide microarrays. Hybridizations were performed on a Genomic Solutions GeneTac Hybridization Station (BST Scientific). A competitive DNA mix (containing salmon sperm DNA, Poly-A DNA and optionally Cot-1 DNA when the nucleic acid population under analysis was human) was added to hybridizing mixtures before hybridization. After hybridization, the slides were rinsed with 0.06×SSC, dried by centrifugation and scanned within the next 24 hours as described above.

As shown in FIG. 3B, although the alien oligonucleotides were present on the chip, they did not cross-hybridize to any known transcript in either the human or mouse universal total RNA set, while the human and mouse control probes did.

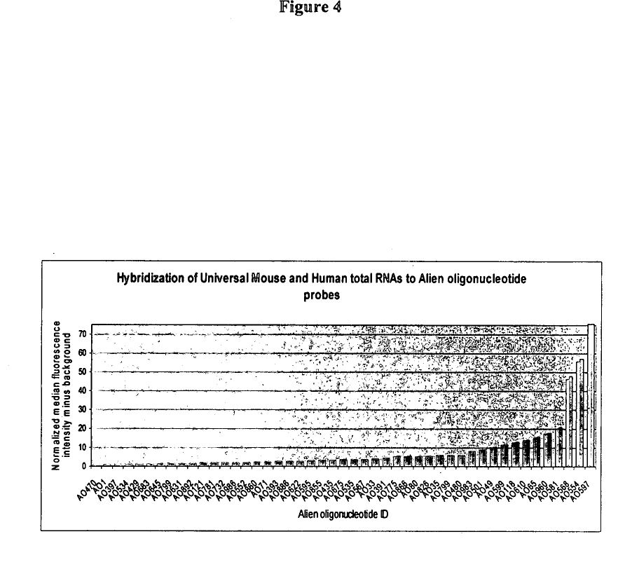

The results presented in FIG. 3 were quantified in different ways in order to evaluate the alien sequences employed. Specifically, as shown in FIG. 4, the 47 alien oligonucleotide probes were ranked according to the normalized median fluorescent signal intensity derived from the hybridization of the Universal Human and Mouse total RNA sets. While most probes gave signals slightly above background, three alien sequences (AO568, AO554, and AO597) exhibited significantly greater levels of hybridization (2-80 fold higher).

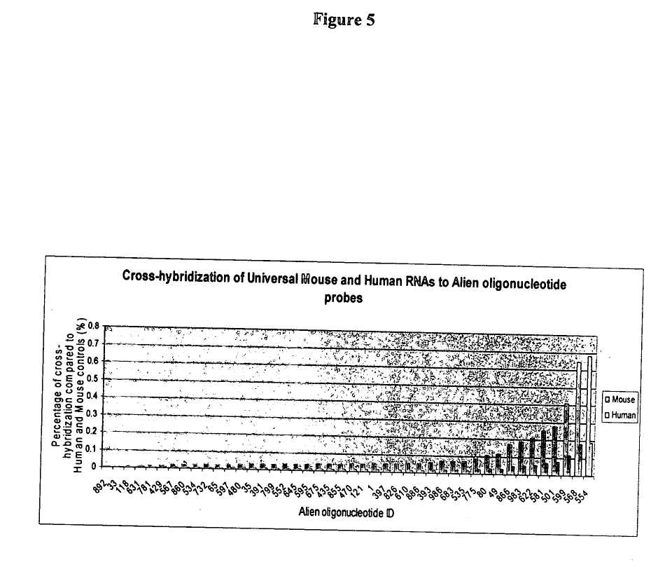

Also, as shown in FIG. 5, the alien oligonucleotide probes generally showed higher levels of hybridization with the mouse mRNA sample than with the human mRNA sample, and no probe other than A0597 hybridized at a level that was as much as 1% of the positive control.

### Example 3

### Using Alien Gene Transcripts as In-Spike Controls

As described herein, one advantage of using alien sequences in microarray experiments is that their complements may serve as an in-spike control, enabling the experimenter to gauge the robustness of the target labeling and hybridization. Specifically, if an alien oligonucleotide is present on a chip or slide, then a known amount of its complement may be added to the population of nucleic acids (e.g., mRNA or cDNA) to be hybridized to the slide. The population, now spiked with a known amount of anti-alien nucleic acid, is then labeled and hybridized to the chip or slide. Global problems in labeling or hybridization are revealed through the extent of alien/anti-alien hybridization on the chip or slide.

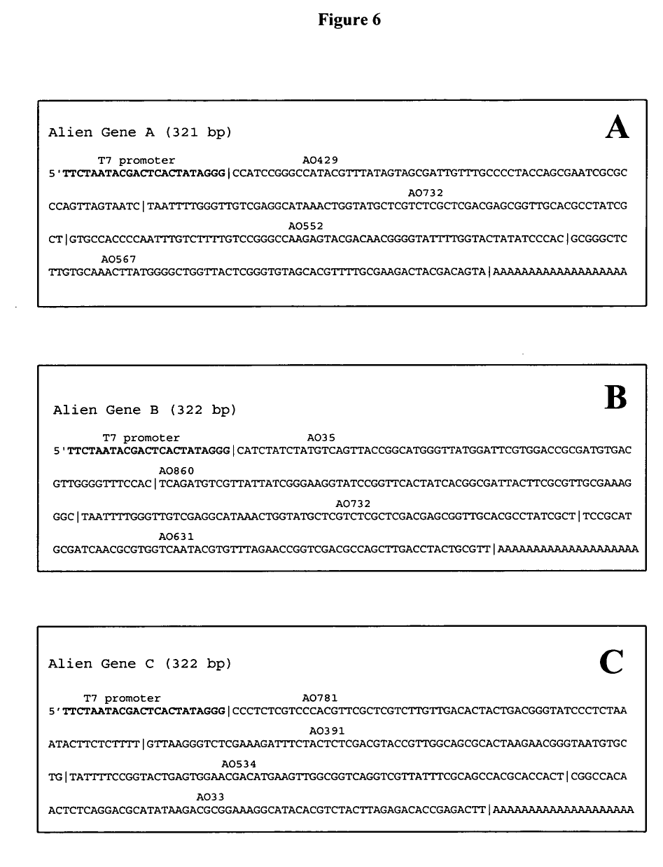

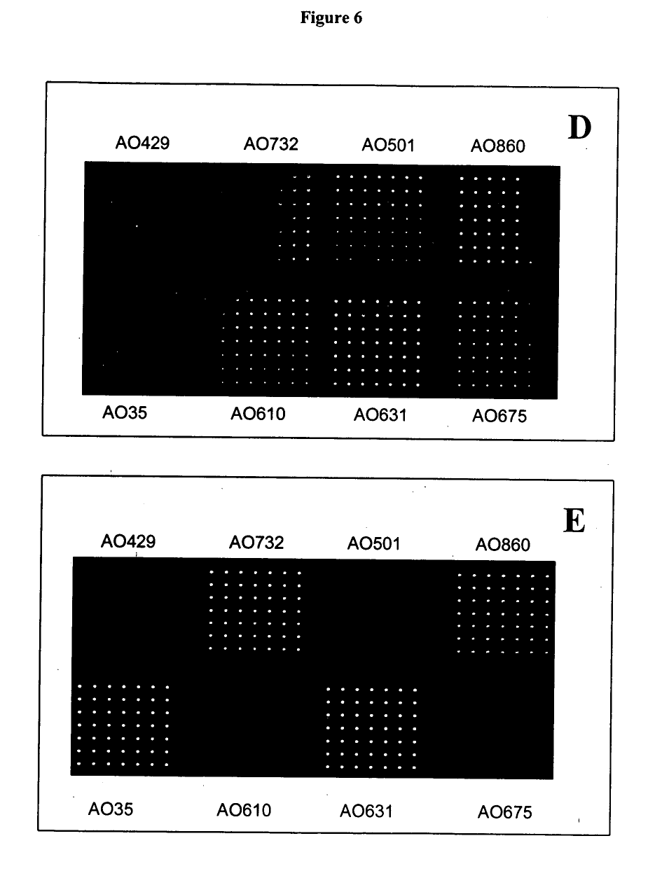

In order to create an in-spike control that would mimic an experimental cDNA sample to the greatest extent possible, three alien genes have been designed to consist of four different 70mer alien sequences linked to one another in series and to a T7 promoter. The three alien genes also contained a polyadenylated tail to facilitate oligo(dT) priming. Alien gene A (321 bp), Alien gene B (322 bp) and Alien gene C (322 bp) are presented in FIG. 6 on Panels A, B and C, respectively.

The alien gene shown in FIG. 6B was constructed, and was used as a template for runoff transcription such that a single transcript containing four alien sequences followed by a polyA tail was generated.

More specifically, 10 ng of alien B was PCR amplified with a forward primer (5′-TTCTAATACGACTCACTATAGGGCATCTATCTATGTCAGTTACCGGC) and a reverse primer (5′-TTTTTTTTTTTTTTTTTTTTTTTTCTAATAACTGAGGTGATTTCCGAC) using the SuperMix High fidelity polymerase (Invitrogen, Carlsbad, Calif.) and the Manufacturer's suggested protocol (which included the following cycle program: 94° C. for 30 sec, 55° C. for 55 sec, and 72° C. for 1 min) was followed. The reaction was performed for 30 cycles followed by a 3 min. final elongation incubation. The PCR product was analyzed on a 1.5% agarose gel and quantified according to quantitative low range DNA markers (Invitrogen).

The PCR product was then used as a template for in vitro transcription. In a reaction volume of 50 μL, 500 nM of PCR product was combined with 200 mM HEPES, pH 7.5, 7 mM NTPs, 20 mM MgCl₂, 40 mM dithiothreitol, 2 mM spermidine, 100 μg/mL bovine serum albumin (Roche, Nutley, N.J.), 8 units RNasin inhibitor (Promega, Madison Wis.), 0.5 units inorganic pyrophosphatase (Sigma, St. Louis, Mo.), and 500 units of T7 RNA polymerase (Epicentre, Madison, Wis.). The reaction was incubated for 16 h at 37° C. Following transcription, the reaction was phenol:chloroform extracted and LiCl precipitated. The pellet was rinsed with 70% aqueous ethanol, dissolved in 25 μL of buffer and quantified using UV spectroscopic methods.

The alien gene B run-off transcript was then reverse transcribed in the presence of amino-allyl dUTP (to allow for the incorporation of a label), using either a polyT primer or a collection of random hexamer primers. The resulting oligodT-primed cDNA was labeled with N-hydroxysuccinamide-Cy3; the resulting random-primed cDNA was labeled with N-hydroxysuccinamide-Cy5.

Microarrays were prepared by linking 8 different alien 70mers, four of which were present in the alien gene and four of which were not, to a slide as described above in Example 2. As also described in Example 2, linkage of the 8 different oligonucleotides to the slide was assessed via enzymatic labeling with terminal transferase. As shown in FIG. 6D, detectable oligonucleotide was observed at each location.

A comparable chip was then hybridized with a mixture of the labeled oligodT-primed cDNA and the labeled random-primed cDNA. FIG. 6E shows that the cDNA mixture hybridized with the expected alien oligonucleotides, and not with the unrelated oligonucleotides. Furthermore, upon analysis, normalized median signal intensities from both the random and oligodT-primed cDNAs were similar for all four alien oligonucleotides present in the gene, indicating that, regardless of priming strategy, all four alien sequences were well represented with no positional bias within the alien gene.

### Example 4

### Alien Sequences as Internal Controls

In order to demonstrate the use of alien sequences as internal controls for microarray spotting and hybridization, alien oligonucleotides were first shown to be able to effectively hybridize with their targets even when included in spots containing other oligonucleotides. Specifically, microarrays were constructed in which a single alien oligonucleotide, AO892 (5′GGTACGAATCTCCCATTGCATGGACAAATATAGTCCACGCATTGGACGCAC CCACCGATGGCTCTCCAAT), was spotted by itself in concentrations ranging from 2 to 20 μM, and was also spotted with a mixture of other 70mer probes, whose concentrationsalso increased.

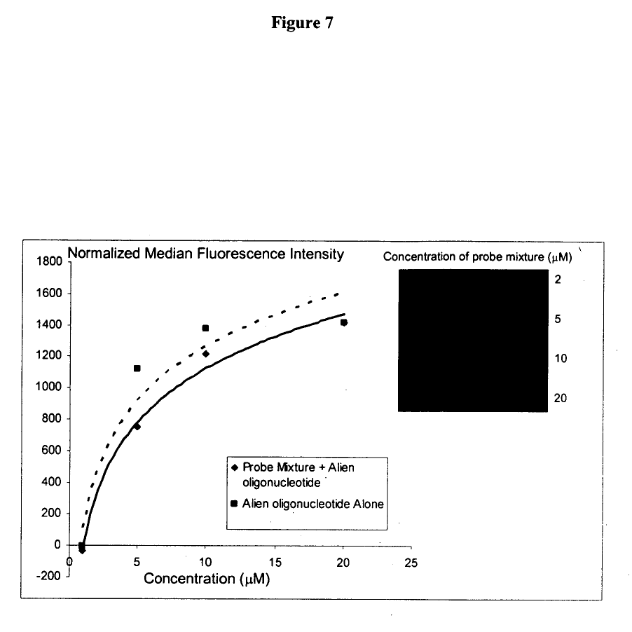

An 70mer oligonucleotide whose sequence was complementary to that of AO892 was prepared, modified at the 5′-terminus with a C-6 amino linker, and labeled with N-hydroxysuccinimide Alexa-488. This labeled complement was hybridized to the array under standard hybridization conditions, and differences between its hybridization to the pure AO892 spots and the mixture spots were assessed. As can be seen in the insert of FIG. 7, which shows one subarray, little change in signal intensity was observed as the concentration of the probe mixture increased. As shown in the graph presented in FIG. 7, there was no significant difference in normalized signal density between the AO892-alone spots and the mixture spots. These data demonstrate that hybridization to an alien oligonucleotide can be detected even in spots containing other sequences, such that alien sequences should be useful in the normalization of gene chip data on a per-spot basis.

1. A nucleic acid array comprising:
a solid support
a plurality of nucleic acid probes attached to the solid support at discrete locations, wherein at least one of the probes is an alien probe in that it has a sequence that is alien to a hybridizing mixture to be hybridized to the array.

2. The nucleic acid array of claim 1, wherein the hybridizing mixture comprises nucleic acids from a source selected from the group consisting of human mRNA, human cDNA, mouse cDNA, mouse mRNA, and combinations thereof.

3. The nucleic acid array of claim 1, wherein the alien probe is present in each discrete location on the solid support.

4. A method comprising steps of:
providing a hybridizing mixture comprising a plurality of nucleic acids; and
hybridizing the hybridizing mixture to a nucleic acid array comprising:
a solid support; and
a plurality of nucleic acid probes attached to the solid support at discrete locations, wherein at least one of the probes is an alien probe in that it has a sequence that is alien to a hybridizing mixture to be hybridized to the array.

5. The method of claim 4, wherein the step of providing a hybridizing mixture comprises providing a mixture containing at least one anti-alien hybridizing nucleic acid whose sequence comprises a sequence complementary to the alien probe.

6. The method of claim 4, further comprising a step of:
measuring hybridization between the anti-alien hybridizing nucleic acid and the alien probe.

7. The method of claim 6, wherein:
the hybridizing mixture contains both the anti-alien hybridizing nucleic acid and at least one experimental hybridizing nucleic acid of unknown quantity; and
the plurality of probes attached to the microarray includes at least one cognate probe whose sequence is complementary to at least part of the experimental hybridizing nucleic acid.

8. The method of claim 7, further comprising a step of:
measuring hybridization between the experimental hybridizing nucleic acid and the cognate probe.

9. The method of claim 8, further comprising a step of:
comparing the measured hybridization between the anti-alien hybridizing nucleic acid and the alien probe with the measured hybridization between the experimental hybridizing nucleic acid, thereby determining how much hybridizing nucleic acid was present in the hybridizing mixture.

10. The method of claim 5, wherein the step of providing a hybridizing mixture comprises providing a mixture containing at least one anti-alien hybridizing nucleic acid whose sequence comprises a sequence complementary to the alien probe and also containing at least one experimental hybridizing nucleic acid, the method further comprising steps of:
processing the hybridizing mixture such that the anti-alien and experimental hybridizing nucleic acids are simultaneously subjected to identical treatments;
hybridizing the hybridizing mixture to the array; and
measuring hybridization of the anti-alien hybridizing nucleic acid to the alien probe such that information about efficiency or accurateness of the processing or hybridizing steps is revealed.

11. The method of claim 5, wherein the step of providing a hybridizing mixture comprises providing a known amount of at least one anti-alien hybridizing nucleic acid whose sequence comprises a sequence complementary to the alien probe, the method further comprising steps of:
hybridizing the hybridizing mixture to the array; and
measuring hybridization of the anti-alien hybridizing nucleic acid to the alien probe such that information about quality of the array is revealed.

12. The method of claim 11, wherein the step of providing a hybridizing mixture does not include providing experimental hybridizing nucleic acids, and the hybridizing step is performed prior to exposing the array to experimental hybridizing nucleic acids.

13. The method of claim 11, wherein at least one alien probe is present in each discrete location on the array.


SEQUENCE LISTING (166 sequences)

SEQ ID NO: 1 - DNA, 174 bp, Alien to Mouse cDNA
atggttggggactgcctctccccagtcggatggtccacctctgcgtacaccccacctgatccggatgaggccagatacacctgtaaggctcctgaccaattcaaaaagacacgcacctgtttgcgatccccaaagccttgcctgtcgataagtgcagaggaactcttaatgtga

SEQ ID NO: 2 - DNA, 651 bp, Alien to Mouse cDNA
atggcctgcaccctggtggtagaggcccccttgtcaaaaactcccgacttgactggtgacttcaatagctccttgtcctggtcttgcctcgacaataacccggttttgggattagtgcagctcaaggtggcctcctcctctagctataagtcggaggaacttgatctggagcttcccaagcgagccaagattctggattcgatcagtggcacttggaaactccatcttcgcaaggagttccgcctcattgtgtgtatgtcgcatgcctggaaccggcggcatgcagctgatttgaaccggtgcaaatggaagggcaagagggcaggctggagaggggcccccgtgctttttgctcccatgcaggtgacgcgcaagtgtgcaccagaccccacagagcagtcaggcctcttcgataactctttcctggatcactaccagagtctggcctgcatttacctaggctcccttgcccgaaagggctcttctctgaccaaggatggaaaggtggattttcagggcccttgccttcgtggtggccagaattattcgaacttttctcagagctcagcgtgttggaaaccgctggacgaccaggaacagatcgcccgtcccctcagtgtctcgttgtactatgcagccttagtgggctga

SEQ ID NO: 3 - DNA, 229 bp, Alien to Mouse cDNA
aatgccaaagttgttaaacctgattcgggcagtcggctgctgtgagaaacagaccctcctggctgccgagagcctcaatgaccgggaggaaatctcctgtttgttccggcgaaacctcctccagggaatgcttctgggagacagagcagatgacaataccagtgaccacacgatagtctgctacaccttcatgatcccctcccacgccaggatgcctggaagtaggtag

SEQ ID NO: 4 - DNA, 174 bp, Alien to Mouse cDNA
atggaagcagagctctgttcacgaggcgtcaacagacgtgacaatactaaacttccactttcgtctttgccttcagcttctcctcatgattccaagagatgtccgcgctctaagatcgctcacgtctgggacaccagggccgacggtgagatcgattcgcgaatcttgtactga

SEQ ID NO: 5 - DNA, 306 bp, Alien to Mouse cDNA
atgaactctctgtctgaatacgagaccttaaggcggaccatgctgcagagctctaacaagtgtaactctctgtgccaaattgtacaaacttgggttgagggtggcaaggccaaggccaatatgaatggctaccagaagcatttggttccacttcgcgttcaaatgtgggagatggcaatgcgacttaatggaacccagccaaatgaattccacccggcagtccagcagtgcatcctggctccttacctaaagactttcctcagtatgcgtcctgattcgcaaacttacccggccaagctgagctga

SEQ ID NO: 6 - DNA, 156 bp, Alien to Mouse cDNA
atgcctcgagggcgtactctggtatctcgtcaagcatggcgaacagtgaccggtaaggcgggatgctctgggcggtatccaagagagagcgggaccttgagtctatcgcatttttccctggggattatgtctaagcggagccaggaggagctctga

SEQ ID NO: 7 - DNA, 135 bp, Alien to Mouse cDNA
atgatgcagccttgctccaaacaagaaagaatatgcggacctcctgactccagcatcgagtccgcgtaccgctcagcctctctcacttctagccctgccacgcttgctccggccttctctgcctgcccctgctaa

SEQ ID NO: 8 - DNA, 144 bp, Alien to Mouse cDNA
atgaggcgagccctggtagtgtgccccttggcgggaccctggaagaaccagcggtccattgccctggtgaaagatcttcccatgaacgccagcgttgcctcatactttatagaaagggggagcatcagctggcatttctcatga

SEQ ID NO: 9 - DNA, 165 bp, Alien to Mouse cDNA
atggggtgggtcaaggccctgcagagtgaaagcggctggtggtttgtattttctcagggtcgagtgagcctgaaacccgagccgggcctagcgctggttgtacaccagggctttgaccaaacagtcacagaatgtctaagcttcacaggaaagcccatgtattag

SEQ ID NO: 10 - DNA, 561 bp, Alien to Mouse cDNA
atgatgagcttcgaacattccgacttctccaatgtcgaggaccgcaagctcttaacggaagcgatgtccacaggcttcgaagtaatcgagtcgccgtgcaagatctgcatgccaagctttggaggtaaaacaactgcggatggcaaactcacttccgtgactcagggcatgaaacactggtctctcaccagagctagtcccccggaccagtcgcaaaagggccgaccctacaggagcacggtgcaaggggagattgaagcgggacagcccccacatgaaatctcctccgactggtaccccatgttcaagatggaaacagacagcccgattaagaatgttccccaggcacacatgggggagttcgggcactgcgacaatctccccaatggcaacacagtgagcaacccggagcctagggagaatgggaatgtggcgccgggagtgggcttagacggacaggaagaaatgggctggctttggccggttcgtccttcttgtatgaactatttctttaaagcatccactctctccttttggatgggctttcttgagcgccgctag

SEQ ID NO: 11 - DNA, 480 bp, Alien to Mouse cDNA
atgggaaaatctcgctttgagtatgcagtgacgccccttcaagcccaagcccgcagtttgggcagatccctgaataaaagcccggtgttcttgttttactctgagactacatccctgccagccaaggatctcccgtgtgagtcaggacttgctgtgagagacctgagcaacaggacacagaacagtctagctatgtttttggcttcacgggggatcaaagaccctgaaatgaagatgaattattccatctatttggggcaacccttgcaagaaggtctgtcccccgtgcaggagaacttttctcaatgggaactcccactcgtggcttacatgagctttttctgtcccttccgtgcgggcgaccggggttcgatccataatcatctctccacggtcagagcgaagattgactactgtggtcagcggtgcagtgcctcagatccaaggaggggccctcaggactattctcaaatgctctga

SEQ ID NO: 12 - DNA, 231 bp, Alien to Mouse cDNA
atgcgggaagagtccaagactatctcgatcaatggtgtgaaatggctcattgatttgccagctgaaaaaatcttcacgaggaactatggtgttgccgactgcaggagaagcttctacatcctgggcctgtttggttgccacctggtgactggagggtaccgaacattcatgatctacatcgggtccatttcttctttcatcatgtatgtgggggtccggatcattcgttga

SEQ ID NO: 13 - DNA, 426 bp, Alien to Mouse cDNA
atggtgccccaagtgtgcgagcagtggagcctgtgttggtcctcgggcgggttcccaaatcctgcaggctcttatttagagccgtggtcaagcgacttgtccagggagcttcagtgccccggctacagcggcttcttaagtggccccacggattttctctctatgggagtgtcatgtcacctagcacaggaatcatttcggttcccactgcaggatgattgcctcctgaccaagatgcacaggttgaaagatttctgggactccaccagcaggtttaagcagctgggcgaatctgaggcccctcagcagattcgcaagaaaaaatcatcgtttagtttctggggctcatcggagaactctgcgcccgcaaccgaaaataccagcaagaagtcccaggattccttctttgatgccatcctcaagtga

SEQ ID NO: 14 - DNA, 192 bp, Alien to Mouse cDNA
atgggtgtgtcgatggccagcttcatgctctcttctggcctcctggatgcagagggagaaagcttcatgtcttggcatctcagcagccctggaacagccgtggaccgaacggcccaaatgtttattcacttcagaatgatggggtcaatcttcagtgttaccctgacgcttgaagtcatgcggtctctgtga

SEQ ID NO: 15 - DNA, 351 bp, Alien to Mouse cDNA
atgacaatggaaacagggaggcacccggtcatgaaggaccaagcccttgacgaatgcgaacggtcgatgtggccggtcccttcttgggcctgggagagttcttgttctcatcgtgtcgatgagggagatgtatcggtactgctggaacagtttcggcaccagactgaacagctcccgcccatgagctactttttggacaagccaaagctgtcttcgttccaggaagagccacggctgtgggtgactttatgccaggagacattgccatttcccctgggtaattctgggtatgatgagcaggaagaggagggcctgtgtctggtctgtccgttgcccagacttcagacatga

SEQ ID NO: 16 - DNA, 153 bp, Alien to Mouse cDNA
atgggtaaaatcaatcacaccacatcgacacctaccttgagcactttaaaaatccccacatttgaggccttacgcccgctactatgccctagactggatccccccacctcgtctgtccgcctggcatttgaaggccagtctcagaaattgtag

SEQ ID NO: 17 - DNA, 324 bp, Alien to Mouse cDNA
atggttcgcaaggttgctcacaatgttctgtatgagaccatgggtcagaaagctgactcaaagtggggaaccagaaagaagcagccacaagggacccgcctgagcaaaccttgcaccacggtggtggagtggctgtctgccttcatgtaccgatcccgcaagaaactgacgagccgcttctatctgaaacctaacatgtcttccggttctatccgctacggagagcggcaaccactctttttggacagcctgctttggtccgacagtggaaagggagcctttgcctcctgcaaatgctcttatgctaaatcattttttgactga

SEQ ID NO: 18 - DNA, 450 bp, Alien to Mouse cDNA
atgagcaactacctccacattcgttccccggagtcggtccataacacctttcctttgtgggtccatattgctcaagcaaagttcggtcacctacaagccttgttaaagcgcgagagtgggtttgaagccaacaccgcgaatgctgggccgctaggcccccgcatcagcgatgacactcgcaatatccttttgactggattgttcctctccctgaccaagaagtgtggatgtgtccagttacagtgtggccgacagagtagcctcgatgccaaaatgccatgtgaccagcactatagaaaggtgcagtctgccctcagccagggtctgcagatgggtggtgcgtgggtgaagcagaaagcaagccaggagattgccgggtggctccacagcagcagccttcaagagcaggccttggatggatcatccaacttcgccactctgtccgtttaa

SEQ ID NO: 19 - DNA, 720 bp, Alien to Mouse cDNA
atgcggagaattaagtttgagttcaagaaaataccttctgttcgtttgtaccggttcttcttcggttcttgggctaagatttctaccctggcatttgtggaggacacctatacctatgccttctggatggaaggagcaggcttcactcttgtctcagctgactgcattacttcccggacctttaggagtccacttgccaaggacccgctggcttggcggctcctggatcttgtgcgggcaaaaactcaagaagcgcggacgaactcagctttgtccttgaagtgctccctgcctgattttggtccactcggggagatcaacagagcccaggcctctgaaggccagcagacctttggctcctttgagaagccgtcagagcatgtcctaacagcaaagaatcagctccaggtgatcataagttatcccttctgctatctgctcatcataccggaacgtccattcgacagtagcaatatgtccttgttcagtaagccaagggtgccggccttggaagtgattggagtacgcctcaagacccagatgctagtcacgcctttcagtgagttccagctatattcccgtgcatttctcagagaatcagatttgtctgagagctccctctgggtgacgatctcttttgacacggcgaatctgtcttatgtccaagcggctgaggaagagtgttcattgagaagttccctggcttacacgtggtcttga

SEQ ID NO: 20 - DNA, 465 bp, Alien to Mouse cDNA
atggggatgatgctcaacttttgtctgagaatctactccagcagaaagggagacgccatcatgtctggcccttctgggtctttccttagaaaaaagagtgtgccctaccaaacctggcgagcggagcagtctcgtaaggtaagcgtgtgctcctcgcagttttactcccagaccatcttgcgttggcggccccaggatgccgaaacagagagacagaggagaagcggcttcaagctggccatgatggcagcgggcaagtgccagcctgtgaacgaccccacctcttgctcttatgaagcttacctaaggcccatctggaatggtatgagctttcttgattggctgatctttgtccccatgaaccttggtggacacagacacagcacctccctgagcgcgaacaaggtcacgtccatttacaaggaatatgcaggctattccacctgctcgtctaccagaggctga

SEQ ID NO: 21 - DNA, 216 bp, Alien to Mouse cDNA
atgcagtactgcgcagctgccgcttccaagctgttcccagccttgccgttaagggcccaaaccctcagacactacctaaatgtggccctacacaagtctgccctcctgggagatctggcctggcggcggaactcggcagggggccagggctttatgactctagggccaaaagagattctgccagctcaggtggccccaggtggagagtttggatga

SEQ ID NO: 22 - DNA, 1188 bp, Alien to Mouse cDNA
atgtatgcctgtgctgctctcagttcattccttgccttcccaaagtacggactgactgccaagagatacccaaccctgagaacctattgcctctgcttattgtggaagtgtgagaagcatattttgtggcaggggatcaatctaacgatgcgacaggtgagtgccaatgggacgcccatggtgaactggggggtgctgaagcccaccactcaccagattctcaatggtgacacagactgtctgtgccgcccgaggtcatttggtttgaaggccaatcaggcccgccgaccgaagaagtaccaaggctgcctctcacggaggtgctctgctgacttcctctgttcccatggggctgttgtaagagatcagtgctcgatgattcaagtgtctttgagcacccggctgccgttctctaatccatggattcaggtcgctgtcatgaagttcttttgttacagaaccaaggcctgcgcatgtaatggggggggtaaaaaagccctatctgtgagttggcaaaaattccagaacttgtacgtgacacggaaagcaatcctagttttcagcatagctaacaagggttccctgactaagataaacatccagcggaagaagctcagtaacagggactcagtgacagagtgcgtcttcggactaacctataggagctttctaggtaaacgccatgtattcgaaggagcctcactcttgacgaacggacccaacccagggaggagcaagtggccctgtgaaacaataagcgatcagtattactgtttcaacaggaagttgtctgagagcggcatgtgcttcatgttgtgtagtacctgcagagggtacctgccgccggactacctgtttgcagctctgctcaagacagtcagccggcacatcgttaaagtccgccaggtgttgctttttttagaactttaccctggctcgaaggctagatcaagcgatgaaattccccacgagcacaataagacgcctgagctggaggaacttccgcctatcaacagctgtacccagattgccatgctcctttgcagccgctcctcagtgaaaaccaaggacagtacgacggcacctgttctgtgttcttttttccttagactgtttgctgaggaaatccggctgcgctcttttgaacgggagtaccgcaaagattcttacaagtacctgcgggtgtga

SEQ ID NO: 23 - DNA, 126 bp, Alien to Mouse cDNA
atggatctcgatctgcggttcattctgttatggaaacaggaggagctggggctgtgtcggtacctgaaaatgagaaaatttagtctgcagtatgggaagacaaaaaaatgttcctcaccggcctga

SEQ ID NO: 24 - DNA, 948 bp, Alien to Mouse cDNA
atgggcagtcgcgccccatcgtctggtgatgaaactcaaatccacgaactctcactcaccccccgggatcccaccttaaaggaggggaccaagaagggccagctaagggcatccccgtacttccttcgtgcaatgccgtccttcctttcagtcaacacaccccaccagcagttctaccaccgtcagcgggccagctttcaggactacgcgggagatatggcctacatcgaacttttcagtcagatcagtcctactgcgcaaagagcactacagatgccaatcaaccctgcgaacgcgggcgcggtatccatggggaaatctttccccttctccatgcttttgcctcgcaactccgtgttacccccaaccaagcgcccgttccaaagactttccattccgcaatctctgaccagcaagggccactacctgagcctgtatctgctggaaggagaaatcttagcaggaaccatctccaccgtagcggtggtgaccaaatggacatctcagttctacatgtgtgtgctggctgtcctttacggtcaacacgcaccttccttcagtcagagggctgttgaggttgaccggaagtcccaatccaaggccccaaaggttcaggaaatgtggcgagacgggattaaattcacgtctggtaaactcctctcctgttgtgaggggcaccgcatcgcctttgactggtccttcccaaccaggttcatacagattggacgtccgggggagtacattgcagaatgcttccagcggtcccggagaaaggctaacttcctgaacgttgacataaacagctgtctgcgcaagagcattgaaactttttttgggagaaactatatgcacccgccgcgcgacccgctctttttcagggtgagtatcccttgctgctattgggcactagagggacccttctgtgaataccccaaattccttcacgct

SEQ ID NO: 25 - DNA, 273 bp, Alien to Mouse cDNA
atggaaccaatcgcgcttaacatcaactaccagcggatgctgctatcggggcatagctcaaaccagatgattcatattgtgaacaaaattgatcttgcgaggaccccctcttctgtaaccagatcccggctcaatgactgtagaggccctttatgcagaaaggaccaaaaggctgagcgcgacagccagcttggcaagcgggtgcactatgcattgatccttcggttcaatcggccaaatgcgcctgacagccaggactattcgctaacttga

SEQ ID NO: 26 - DNA, 198 bp, Alien to Mouse cDNA
atgcggaagtcgctttcgcgcaaactgcggatggcctgctccaagggcctctccggggttcctgtctcctcttgtcacatgcactacttcgacgggtccctggtggtgcggctgacctgtaagaggagacatggcttgtgcaaagaacagcagggtatcgcgggcaccatcagacagaacggcaccatcctaagttag

SEQ ID NO: 27 - DNA, 213 bp, Alien to Mouse cDNA
atgtattatccagatattacgtatcccaagcccagcagaattattgagaacttagatgaaattgtttctcagtcaggatcgattgaaaatcactcccgaccgatgattggtctgcgtgtcaactctaagtggatgccacttggagggggcccctacaagatgatgcgaagcagtagaaaaaaggtgagtcagtgccttctgaatgacatgtaa

SEQ ID NO: 28 - DNA, 675 bp, Alien to Mouse cDNA
atgggtgatgtggtcatgacggaggaaagctgcagcgccttggtgtttgaaacatctgcaatgtctgggttttacaagacatggacaccccggttctacggagtgcaggggcatcgtgtctcggacctcgctgctgttcaacagccggcgcgcggtgagtttcgaaggcacccttcaccctctcaacgactgtgggcactcctgggtgcatggtggcgtggatctggcatcctggactccggggccctgcgtgaaatggagctgggcatccagggtaccatacgattctggctacctactgcgcgctcgcggagttgcttgctctgccgatgcctgggggctgagatccaggctctcaagggcaacaaccagaactcattctatcgtcagctcttccgccaagcttcgtaccgttatctgagatgtagtttggcgtacccatcgatgggtgacttcttgccattgcagcgcggcaagtgggttctcctgggcagagggaagcctccagggcaagctcgagctctgaagcgcacaggggatggcaaggggcaggctcgattaagaacaagtcaacttgttcattccctgggagagtatgtgcaggttttccctttctatccagaggacctaatgctgagtaaagaccaggaagacagccaacagagagtgaactag

SEQ ID NO: 29 - DNA, 609 bp, Alien to Mouse cDNA
atgtcaagtgaaacttcaccccgcctgatccctaagtcctggagtagagggcgcagcgaaatttcaatcccttccatcattgccctgggtgagctgcttgcccgttggaggctagtttctctctccattggcaaacgtcttatgcatcctctgcgccagacatacatgcgaatttttccacgaacctttattgtcagtaagatccctgatggcatggagatcatgctaagcaagtggtatgtggctaatggaactcccgagcccaagaggttctgcctgacaaccagtcaatggctgagcctttacatgatttccccatgcacatcatactgcagactccgcgcatcagcaatgccgcgaggcaggcggcttgaagcctggcacggactgagcaaggctgccaaggagatcactgcatctcggatgtatgcggagatcctcttgtccgagttaatgccggtggagacttatatctgttacttcccgaacctcgaagccagatgtccacgaaaatccccgttttcgcgtgatgaatggagcatgataagcgtacctttgatcaacagtgtgttccgcttgcgcttctcctggcttgcctctgggccttga

SEQ ID NO: 30 - DNA, 789 bp, Alien to Mouse cDNA
atgttcacattcaccagagttgggtggcctcggtcccattggagatccgccgtggggaacagtgaacgacccctcttcatatgggcagccggtgccctgcggcccaaggaacctcttctgtttcggttggaaaaaggccggggtgtggccgagctgcggagaaggctgagatttttacagtgtgaagctatgtattcgaaatttctggggatccctgaaatgatggaaaactccaaggccgtgatcgtcaatttttgcaccaaaatcggacgcagggaatgggagtcgcaagcgtcaatgctcccacagctgtcaaatttcatgacaccgcccagtgaaagcacgctaagcagctcagccactttgaggatgagcctcctgtacttcgcttctgcacccactaacaagacaaaaattaagggtgtgaatttctactcgcctcccaaccacatgccccttaagctgctagagtgcttgagacatgtgaaccgcgagtgcttcaccaacctgggataccttctggcttatatgaattgcagcatggacatccttaagggcaagatttctgacgtgatgggaccgcgtgcctcagaagtcaactcaacagacagtactatgtgggtcctgtcaacaggagccacccccaccgtggttctcatggaaacaacatgtgcccccctgtcttggagctacctgcctgctctgtatgatgcaccgcgcttcacatccgaaacctacatctcccttgctgaagcctgttatcgaagccaggcctttcagcaaatgtaa

SEQ ID NO: 31 - DNA, 258 bp, Alien to Mouse cDNA
atgtacctcatggcactgaatatagagcctgaagatctggcgggattcagcaaactcactatggacctgtattttgatgaatatgcagattccatgttggacaagagtcccggcctgatcgaatttctgaccgttgggactccgaagtgtcttctggggcctcggctgagtggtagcgatgcccatcgggccagtatcgctcgggactatcgccccatgatccaacaggtgggtctgggtgtcaacttggtcacatag

SEQ ID NO: 32 - DNA, 264 bp, Alien to Mouse cDNA
atgatttcccacacaatctccgagatcctcaccgaagttcagcggcagttcttctttctggcctgcaggggcttcttctatccgcctctcatgggtggccgtgaagcttctgaaactcagggaatggaatacggcaaggggtggaacacccatgtccagtgtcgtaagtgcaatgattgtgtgtgtctgttgggggaggtttatgagaaaggcataagatacagttgcagtgtgagttacagatccctggcctacctgcaatga

SEQ ID NO: 33 - DNA, 210 bp, Alien to Mouse cDNA
atggaacctatgtctgcattaccactcgagagcgcattgaatgacaaaaagttcagtaccaagacggggttgccaagcggacttaaatttggagaggttgctccagcccgagcccccaatggcttgtctaggaaagcttccaccaggttccaacagacggacgttcgtggcaaccagcagcatggtcttatcatgatgcagatttgttga

SEQ ID NO: 34 - DNA, 375 bp, Alien to Mouse cDNA
atgcacggcatccactactcgctccccacccagactgctgacaaagccttaggtgtgggcatttcctcccaaggccagattcctcaggcaaatgctggcaacctccccttcgccgatgagccgggatggcagatgctcaggatgggtggtggagaagaccagtcccggttcacaacatttgtcttgattcgattctgtgtaatcttcgtcggcaggtgccaggatatgtacctgctcaaaacaacgccacctgaactgcgccagaatctcatgtgcctgaagatggagtgcactagcgctctcaagcttaaggatgcgcaggtgcagcttgacctcacgcttcccttttgctacgccgccacggtgtcggcctaa

SEQ ID NO: 35 - DNA, 135 bp, Alien to Mouse cDNA
atgtcaagcttcaactcacagtacttcttcttcgcactggaacccacgtggtggttctctatgggacctgaggacattgtgatgcaccagctcctctcttttttcaggctgtgtggagctgccagttaccggtga

SEQ ID NO: 36 - DNA, 231 bp, Alien to Mouse cDNA
atgtgccagagggagagacgattcacatacccgcagattagccactgcagggaattctgcagaggcttcacccaaagtaaagaacctggaggacatgacacagctgagtacaaggatctggctgaagccctgccaatgaagaacttcagctgtccggtgctggaggagagtttcctttacgcaagcgaaatgagagcttttctcaagcagcaattcgatagttggaggtag

SEQ ID NO: 37 - DNA, 180 bp, Alien to Mouse cDNA
atgtcctgggtgctcaaacagtttaaggtaatgcgagccagacctcaattcctgatggcaacttcaacacagggggaatgcaccaagaactggaatgtgaggtggaaaatatgggatctctcaatgctgcttgactctcataacacctcttacttttacatttgcgatccggtagtttag

SEQ ID NO: 38 - DNA, 123 bp, Alien to Mouse cDNA
atgcattggtcccaggtgaaactgttggagcgcttcagtaatagcaaagagacgggtgctgaagatgtgctagaaaatgccatgccttctgaaatggcctctacccttggagaaagcccctag

SEQ ID NO: 39 - DNA, 147 bp, Alien to Mouse cDNA
atggattcgcccacgacattcacaaagttcacaaactggattttcctttattctgtgagggacgaccacgtgtggctggtatctccattccagcagttctgcttccccttatcctctgccgcacctgggccgctggcatgcaattaa

SEQ ID NO: 40 - DNA, 339 bp, Alien to Mouse cDNA
atgagaaaggatttggagtgcctcctgtccaaaggcacatcgaatatgctgaagagttttctgatctgctgggggaaggctaccctccgcttctgcgaagaaatgcctctcacccttgagatggttcacctctacatggacatccctgatgaacgctggcctccctctaaccagccattctttggaaagttctactcgactttcttcagccgccacagccctgggcccaagctccaccgccctcagggtgcaggaaggacacagctgtcagaggtcgtgggcaacttgcggtgggatcaatactgttggggcaatcctcaaacgcgcaggcccagttga

SEQ ID NO: 41 - DNA, 354 bp, Alien to Mouse cDNA
atgccctgcctgggccgacaggaactcgcccgcgcgggaggtgtgccaggaagtgcggatcggaggaagaaagcgttcaggttggaagaagccagatatcccctgtacatggagggtcttggatctgagacgcaaggggcagcaaaggatcaggccccctcgttccggagcccgagaatggccctgccctacctaagactccggcccatcaagagagtccccatcatctggcggatagtttttcagagcctccaccctggcgagaagcccagggagacgtatggaaacgcataccggggagaagcggccagggcagagttcacccaagagtctgcaagccaaagcttcacttga

SEQ ID NO: 42 - DNA, 267 bp, Alien to Mouse cDNA
atgaccttcatgaacgtatgtatagccgggcaagatgcaacgcagccatattatagggccagttacaatagccacagtaaagttcacaccttggaatgtcgagttgagctcaaactcacagaattaatgcgctgtgcgcatagaggaaagggcacccgtaccacgcgctgtcttatcactgccgccttaattctgtgtccccccacctccaaagaattcgcgtacaacaacttgctcattgcttcccacacttggggcaatgattag

SEQ ID NO: 43 - DNA, 210 bp, Alien to Mouse cDNA
atggcaccggacaggtccacattctcttacctgtgggatcctcaggatcaccatcaggacgcctcccctagttctccaattgccagggtgtcatcacctgccttccggggttatgactcagaggacctcgcatgcagccccccctttcagaatgcccagctttggtgcaattcgagaaactcaactgtaatgctgtacctcacactgtag

SEQ ID NO: 44 - DNA, 942 bp, Alien to Mouse cDNA
atgagcgtgagggaacgtgaggcttcagacaaatctttctttttggtctttgcattttttttacgaagcagtttcattgggttcatgagacagtctttgcatagctgtgcgaaagcacgctgcgcgacgttcaagccccaggaacgaatgtgtaaccagcggaccatggttgccaacgctccggaacccaggctgatgacactggttgtccgcttggtcggccatggcggttgcacaatagtcacttctgacccccgatccccccagggtgagaaggcccaggatcgctacaacctcattcgggtgcccctgtacccggctgcctacatcccctgttactacatgaatgtgctatccatctcaagggaacttgagctgctattgagctcaatccaggttgaaatgagacacccagtgagcaacccgggacagttatactatatctctggtcaggtggatcccggctgtgacaggagaattgccaagtcgcctcgggatgaccagtcgggatctccccggcagagagatgcacccagctacaaggtttccacgttttaccgggctagcagagctaagagtagactaaaacggacagaccccaagaggacctcatccagtcattccacgttgattttgtttatgctaatcttggacacttcgaagttcatggtgaagtccagccggactttcactctccttcttcaggacttccattcagtgacacggaatcagagctccagatttcagttcaggcggaatcaggaaacagcgagatctcctggagtggccactaaggagacgggagcgttgacacagatgtcacccctttctccgcagtaccgcagagtgactgagtcgtttttcttagtgcacggttctctctctccacgtcggtgcctggagccctaccctttagcccaactggaggaaatccagaagtga

SEQ ID NO: 45 - DNA, 357 bp, Alien to Mouse cDNA
atgacctacctgtggatgaaggcgatcagcagtcatgccaagctgccggcaaacttcacgatacagtcattctcccagtgcattcaggaaacaaccgcaagtcctgatagagaactcctgacgatgctgaagcccacaagatctcaagaagagacggacctactgaatagactgtggccggataacctctcttctctgacggagatgccaatctcccgttgtctgtgcagaagcatccgcccttacacctcttcagcggactccgtgtctaaagagatgtgccagttttggcaggtggcctttggcgaggctggcaagcgtgaggactgtcctctttaccccaggtcaatcctgtaa

SEQ ID NO: 46 - DNA, 129 bp, Alien to Mouse cDNA
atgaaatcctgcgtggatgaagaatcaagtcattgctatgggtccgcgcggtgggaagcgcttaagcagagcacgggttttttcgccactcgtgagcgagagagcggcttcaagcaggatgggtcctga

SEQ ID NO: 47 - DNA, 156 bp, Alien to Mouse cDNA
atgctgctgatgccagagttgttagaaacaaaggactcaatggaagccgaatccaaattgaagagcatcagcatgcagaaggctgagttcaaagaggggggcatttctttaggaaaacggctcacatcgtacccgaaggtccctctggaatcttga

SEQ ID NO: 48 - DNA, 240 bp, Alien to Mouse cDNA
atgttcgccttcttagatctgactagtttcattctcgcgggccgggcttggtacactacctcaccctctcctgacaccgaaatctggcatttaccgccttctggtgctgagctgtgcaaagcttgcctcttgcgaacccgcaatgcgacaacagactctgagtaccacactatttcccggaagtacttaattgaccccatctcacagctttcgctgtttaccttaatgcacctgctctga

SEQ ID NO: 49 - DNA, 138 bp, Alien to Mouse cDNA
atgatgagcaagcatcacaccccaaccacggtactctgctgccaaaatgaagacctgcagggaaccccgaggctgcgagtgctgaacccaaatcaaaatacctggggcatcatcaacttggcctacagaagcatgtga

SEQ ID NO: 50 - DNA, 201 bp, Alien to Mouse cDNA
atgaacgacatgcatgcgctctttgcgaccaaaacacgtatcaccgagaggggaaataagttcttctcccagccctcgaccaactggaacacgttccaggcagaggagcactgtcagtccctcagagcgccactccgtaccagcggtatgtatggcccctcatgctcagcgtacctctttgatatacttctgatctcgtga

SEQ ID NO: 51 - DNA, 240 bp, Alien to Mouse cDNA
atgatgacgcttggttttgtggaggcccaaatccactctttacctctgactctgagcgtcctctgctgtttgaaaatggatcagatgggatccattgagcctgacagaaagaaaaccccagagctcgagctgatgcccgcactcttggccccgagtcgtcagccaaagttcctgccagcggcggatcttctcccagagggtgctcagacgtctaccctcctcctgggtcaggcaggttga

SEQ ID NO: 52 - DNA, 123 bp, Alien to Mouse cDNA
atggaagagaatggcctggcacattcctacactggggtgaagttacgggccaatgacactggctccctggcgctgcgtaagcagtcagatgtctgtgttgagtcccagacagcaagtgcgtga

SEQ ID NO: 53 - DNA, 156 bp, Alien to Mouse cDNA
atgaccttgttcctttccggcctgtaccccaagtgggccgtgagccagagccactatcaatcctgggagggacccgacatcgctgaagggaccatcgaggatcacctggagcgcctcaaaccggtcatgagagccttgattaatggtgggacgtaa

SEQ ID NO: 54 - DNA, 225 bp, Alien to Mouse cDNA
atgacacagtactggaggattttgatcgtgctgcgaattgatctgccggtctccttcctacagttctatggagagagcccccctcagtggttttgccgccccaaacgctgcttaaaaaggtctcggtcgaacggactaaaggcacgatgcaattggccccctgttagctctcgcacctacatcaagttcaagacaatgtcctatgctctgaagtggacaccctga

SEQ ID NO: 55 - DNA, 882 bp, Alien to Mouse cDNA
atgattgtgttgaagtacatcctcttgctgtgtatttacataaacctcctggggtgcagaaatgcaaagactagctgtgagtgtcccaggccgaccattaggaagtatgtcaggcagccttcaatctcttgttacatgcactggtgctgccatcggaacacaggtgagcagactgacagtggtcttacacccaggcatgatcggcgtagccctgacatggctaagggtcagcaatgggttgtcccggcaatgggcagttccgggggccatgagccgaactcatctgcatacttatgctccagaggaatatacttcagagaccggaatgaatgtgccgagggcctgctccacacttggcccctggtgtatgacttcgtgatagaactaacacaacggttcccttacaactcctcgggtcacggcattgaagacatagaatccttcaaaaattggaacttgtaccggactttcgtcatctcggagggctataaactactgaacatcaagagatcaccaaagtctgagttatgctcaggacgtatggctttttctttcctccggctgtttctgttccacaagagacagccccgtggtaaaatggcaatgcgctatgagggcaagtggatctttcgtggggaaggcacagagagtggcgttgtccctctcagggtcggactttccaagagcgcaggcaaagataggatgtgtcagacccccatgaccttagcaaccaagggtcgaaatacccgaggcctgcagggctaccgcctcatcaagctgaagtgtgctcacctgtgccggatggatgatcaggagagggcggtccgggccatggccatcccattcaatggcaagggtggggtgacactgtctatgctgtaa

SEQ ID NO: 56 - DNA, 264 bp, Alien to Mouse cDNA
atgaagctttgtcctatgaggtggctaggcccgaacaagccaaacaacctccacctgtatttgccgcctatggtcccataccgccacggattgaggtgcacatttttcaaggccgacttctgcagggacccctgttggacaaatatgtggccaatcctcaggcgaaatctgattgcgcaggcagggctgtactgtccgtttcaggtcccactcctggagatgtctgatttctccgctaaccgagaagaaatctgggctgcctga

SEQ ID NO: 57 - DNA, 327 bp, Alien to Mouse cDNA
atgccggttgcgcggtatcccagtgacagtctcaaactgtctctgaaatccaaggcctgggtgttccatcaaaaccctactgggcccttcacgacaacccggcccgtcggccgcctgcaggggcggcagcagcccccccttggaggtcagaagaagttggccgaggagcatcctagacgctccctggccaaactgaaatcggctggggcgagcactgggggacttaatattggggatgatcggaccttcccgctgtgcacgtcggcctcgctcagcagacccctcaaccctaagagtaaacagagcaacattatttgcatctcctga

SEQ ID NO: 58 - DNA, 225 bp, Alien to Mouse cDNA
atgacaggtatcttttgctcttatgccactaaagctggaactgcaatgtccttgagattgccccctgtaaaggccagcaatgcctgtgacctgagccctggaacatgtcctcaggacctagatagtgaaatgatcaatcaccagtattggaatcgcctgcggcagattcaatgcggtttgaaatctattgacatctttgtcaaactaagaccttctgtcagctga

SEQ ID NO: 59 - DNA, 339 bp, Alien to Mouse cDNA
atgaaataccggtgcttggggcagctcactgcctcttacaccatggcggaatatttggcattggcaaaaacaggattatttcccaataggggttttcctcgcaagacagaggggacttgggagtccagcctgcctcagtccttcgaagataggggaggctcaggacgcctgacctcactgcaccagttccctgatgtgatggccaaagaggaccggaaaaccgaggactttgcggtcagctctctcccagagatccagcgcgtctccacgggccggccagatatgagatatatgccggaatacattgataatggccccggcagcaactgtgtgttttag

SEQ ID NO: 60 - DNA, 321 bp, Alien to Mouse cDNA
atggacggagactcccactatcgcacaggggggaccaagcaggataccctggtccagtacacattgctccctgaaattgactttttcggggggattgctcagaatatgatgatcatgcgagttgccagaacccccccatttgttgcagaacaccgtcagcttatgcaggatggagggccagagcagagaaatatggaggcccgtgaaccagcccaccggctcactaaggcgatgtatgtgtcatgcaaagcagaagtcaaggggatggtgacgagcctctctggggtgccgacctgcggcctgccatcggaaaaggagtga

SEQ ID NO: 61 - DNA, 192 bp, Alien to Mouse cDNA
atgcagatgattgtcccaagtggggagacaaagatgtaccctccgctggaggccctccaggaggatgactgtatccaggcccagtggctgcacacaacctcccaaagcttccatgagttagtgttaaggaatgcagtccgcacaccatcaaaggttaccaaattcccttgcaaaaagttctgcgtcatttga

SEQ ID NO: 62 - DNA, 666 bp, Alien to Mouse cDNA
atgagctgcccttttcttcttcgtggcattcagatgccttctctggagagaaccttcgtgtcagatcctggctattccatccattttggatctgaaatgcttgatgttgctcatcttgcttctggcacagagcaagtccactgggcgacactagaatgtgactcgcagctcggaaggacacttgagcctcttgaggagatcactctaagttgggtgttgttcctcctcaagttcttttcagaagacatctggaaacttaaatccaaagaacgttccggcgatgacatgcttgagaggatcacatcaatggagctcttgctgccactgagacggctagaacagctaagcttctattccttcttctctcagtgtactgcccttcgccggagcaagaccagcccaccaattcctctgtgcgtgtccctgggcagttgccataagcagcaaagaacctggctgtacaatgcactgatcaagtacggggcttcgaggagaaggaaggtccccaagcggatgcccattgagagtccgttcagccttgatgaggagtgtcttccattttcagtaatgcggcaaagggagacacggacaattggcctcacacccatcatgcagttcctgacctgttcgcccgtaaagagtgtggatccgagccggagggcatga

SEQ ID NO: 63 - DNA, 1311 bp, Alien to Mouse cDNA
atgatcactgccaaagatgagaccagatgtctgcattcctcccgagtagatcggtatcggacacttgcggacccgatgtctgaggagatgtcgtgttgcctcctggttgggcgcgttcacgccaagggcctctttgacaaaattgtcctaatccagaatcccttcatcctccacgactttttcatgcggttcccttctccctcccaggtacctctatatcagcgctacaaacaagaccttgataaggacctgtgttccagcctgccttggtactacaacccgaagctgcggcagcgcacttcgcagctcacctacaagctccgcacaatctctgttggcccaagacaagaccatggcacgaagacgtctctcccaatgctgactattacccaggtgactgcactgagcgacctgagaatttttttctctggatttggggaggacctccccctggagccctttttctcactcctttcgtgttatcggtgcgctttctgggttttacagttcctgctctatacaaggaatggcctcaagtacagcaaggcgcatgacaaagagtgtccatggcccttcatgtccaacttcccacatgcccgggcctgtcggggttggctgttttcgtgcttcagaaagacaagaactttaccctcattcgacagcgtgagggagatagtcttagcctcaaagtcctccgataggtacatgaagcattcagtgcatcggagctgcagttcaacagagggtgccgaatccaagacgagcctggactgtcttaattcaatgcagaagaagaagcgtagagatgaagaattactccaaacaaatgaatttatgatctcctgtggatccctggctgtgcaataccgaagcatctccggcataatttatttgctccgggagcagcattacatgcaccagacccgcaccagttttcagtttacccaggaccaatcgttcctggctcgggagaatcacaattgggggggtgcctctaatgactacctcctgcgcgagaagctggatgggaagccaatgagaggcatgatgctgtcccaacacagcgtggcatgtggtttgcagggcaaacccattgcaaccaacctgttcaagccttcagtgaacttggcagaagagttgtctgtgaaatacactggagctttcctgcgctcagacgccctgctacagctggctcaggccggactgtggccccagaagccgtacctgatttggagaatcagggtggaaaagacccacgaatggggcacgggtgaactggcgctgagcatggtcctgagctgcttagactga

SEQ ID NO: 64 - DNA, 306 bp, Alien to Mouse cDNA
atgtgctatccatcgcctgactggagaattgtgataataacccagttactgaatacgagatggatcgcagtcagggcactcttcatggcaagtggacgcaagccttgttcaaaggtgatccaagccgccattgcctcaatggcacagctgctctatgtgtcaaaggccagcacattagtagggtcagtgatggagggaagcgaggactgcagttgcgagtttcctgatatgcctggtattatgggagatgtcccttccccaatgttcactcttggcatgatcctgccattaaccttgtttcaataa

SEQ ID NO: 65 - DNA, 264 bp, Alien to Mouse cDNA
atgctgacactttgcatgatcctccaggccccgacaaagagaatgatggatggatctgaaagtggagtgttgcagttcctgcggagtcgctactcagggtacctgggagatcccatggcatttctcgaggatgattccagaagtaagccgacggagagaaccggccttcctgtggagatccacatgatgtcgtttctggaataccatggtgaactggtcaacttcttctggcgcagaaggcagcttcaggacgaaggactttaa

SEQ ID NO: 66 - DNA, 285 bp, Alien to Mouse cDNA
atgcacttgcactacgatcgcatgttatttatgcagcacgaaacgttggttatatctatttcgcagatcaatgacctctcttgcaccacgtcaccagccacgatgggcaggtgcataacctgggggcccacgaggacaacttttctgctctttcgggagactgatgtcagccacctgtgtttgatcaaacagctgagcttcttcagtcagatcctgcagtacaagcagctcatgtcgaacatatcggagcgcacgggacgatacatcagaagctaccatctctaa

SEQ ID NO: 67 - DNA, 663 bp, Alien to Mouse cDNA
atgaggcactaccctgcttggcaagcctcagccatgctctttgagtacactggggatggtctccagcagtcccctagtcttctgagtctgggctcaattgccaatacggtgatcatacgaacggaccgggccccacaggagcgaacgtcctgccataatggtgaccttatcaagagtgccggcacctccctgctggatatgcgagatccgcatgtgtcagcggagggagtgactccctcgaacctgatgatctgcaagactccaccctctggtttctgcctgtctcactcggactgctctggagaaaagcagatggctctgagaatgtcagccagcaatatctttcagggtcggaaaaccccggcctctccttgccagtcgacagctacctgcattctctggtactccacctcaacccgtgctgactatattcggcagttttacctgtgcacccgagcgaatgggcgagctccccgccagaactgcattggcatgggcatactgtcattgtattctccggtccagatcgactcccctccgccccagtgcccaacacccctgttgagcctggtcggccgggtgacgagggagtcacagcaggttggggtgcaacgagccctaatgctgggtacgagcacccctctgctcaaccgccgcaagtaa

SEQ ID NO: 68 - DNA, 120 bp, Alien to Mouse cDNA
atgcggattgatgaagggacccaggaggagtgtgagctctgcgctctgggcacgaagagcccagccatcatttcgcctcgacagtacagaattcgaactgtgggtttcatgctcagctga

SEQ ID NO: 69 - DNA, 249 bp, Alien to Mouse cDNA
atgctatcggaggcctcgagagatcgcgtgacggaaatggccatgatgacagattcttatcacctgccaaccatgcctctggcccctgagtactctggcacgtttagggaaagctcttggcgaacatctccacatgcgattgatccaggctggcagagccaggtgtgtgagcagcatgataaccgcttgaacagggagtcaatcgctcaggtcgcttatcagagagggatctggatgagcaagaactga

SEQ ID NO: 70 - DNA, 438 bp, Alien to Mouse cDNA
atgtacatgccgatttacgagcccaagatggagatgtccggtcagcccagaatcgaaaaggcccatcgggatggcaagttagcgacccagctctcttccgaatatttcaccgagaaggagctagacctggttgaccatgctgagtcttacccaatgatagtgggagattttgggggcacgcccaccaagaattcaatacagaccccaggcggatcgatctacggcctggctcagagggacatcagctttaaattaatgtccatgtccagcagttggaagaatgtgggaaggtatgcagcccccttttgcttaggtctctttccgcactacgggaacatggaactacgggaacttctgttttcccacatgaaagcgcgcgaaaccagaaccacgtcaaccgagtctctgacatccatcagactcaggtcaggctggtga

SEQ ID NO: 71 - DNA, 489 bp, Alien to Mouse cDNA
atgctgagatacagccggatggccatcaagcaacagcttgaccaggtggtttacacacggtccctttcattcacggacctccacttgcagaacaagcaggcaggccctgaaaaacatggtaacttcaacctctggggccgcatccgggatctcaggatgcggtgtatcctgaagttcagctggggaggagaggtttttgttcttcaatcaagttgttcctctgactctttctcagttgagattgagttggcagaggtgagattcctatcctaccagaactcacggttgccagcgccacgcaccgactatctgagtgcgagccgcacttctaaaacaagctgttctctgcgcgtgttcatattgggacaccagctaaactgccctctgtgcactgctgcttcttttattgaagggaaactatgtagcaacgatactggagactacagctggccgcaagcgggcccctgtaactggtccgcttatctgtaa

SEQ ID NO: 72 - DNA, 303 bp, Alien to Mouse cDNA
atgattggaaaagatgagatctatatgctgtcaaagggacatcagccaagacgtaggactctgaaggcctcaacccccaacctggtcaggcccaagccgccctgcaccatctctgtgcgggccaccttaatgctaatctggtttcccttccagtgcctgatagctaagatgcagttgaccctggagacctggtctccctggattatctggctcaatcttaagggatggccctgccggatcctgccgcttatgtacccatcaagaaagtctgcagctgactacactgactctgtggaaaactga

SEQ ID NO: 73 - DNA, 141 bp, Alien to Mouse cDNA
atggggctctggcggaccctgagggccgatgtcaagaacagcgatccatcccctttacagaaagggacgaaagctaagcaggtggagagccggaaaatcatggagtacgcgcagacagaggggcacatcacgttggagtag

SEQ ID NO: 74 - DNA, 180 bp, Alien to Mouse cDNA
atggctcggaacctcctgggaacaggacccttttcgcacgaacgccggaaccagcaaaacgctgagttgggaactgagagtattatccttctggatggagataggagaagtgcgcgcacatctggcaagaggttcaagaaggtatcttattacttccagtgtgactgcctgacgctgtag

SEQ ID NO: 75 - DNA, 141 bp, Alien to Mouse cDNA
atggagcttccccgctccagtaagcctatgaccccgtatcctgagcgcagcgggatggggcactggtggattatctataccaagcattcctccagagggtcctctaatatgatctgctgtggtccagactctagcaaatga

SEQ ID NO: 76 - DNA, 123 bp, Alien to Mouse cDNA
atgctccaggaccgctgcttcctcgcaaagtgcctcttatccagcatgttatgctattacaaaaaaggcttgagcgaggcttttggcgaacccaatgaacagagctgcaacatgcggatgtga

SEQ ID NO: 77 - DNA, 177 bp, Alien to Mouse cDNA
atggaacaaggacctgccctggaggaggaaaagtcagcttgccagagcctgaccttcacgtttctgagtccctcgagaggcaaccagatgcagtggaactcccaggttggaagaaactggactgtactggtgccaaaggattgtgctagtgtgtttaagagttccatgaacggctga

SEQ ID NO: 78 - DNA, 174 bp, Alien to Mouse cDNA
atgcagcagccgttcgccagttactccaccagtttcaagtcaagtgatctggcgactaactccagcacgcagctggtctgttctggccatccctcgggacttcccttcgcttcaatgttcattagggctttgtcgccccctgcgctgcgtggccccccaaagctcggatcatag

SEQ ID NO: 79 - DNA, 363 bp, Alien to Mouse cDNA
atgctgagccggtttcttaaggcctttctgtttcggtgctttcagtgttctgagcgggaaaaggtggtgaagaagctctcaaccatccagattgagaaggaggagccgatcgccctgtcttgtggtaaggccccccattctgacctgaaccaagtgctccccatgtttaatttcgagttttttcatgggctcaacgtggccgagaacctggtgtctggaactgcttcgcaggagaagggacaatgctgctatggtttcaacagcaaaggccgctctgtccgggcactggaattcgtgtgtatcagggccttcagcaacatccaatcggatgactccagtgacgccccttttggcctggtttga

SEQ ID NO: 80 - DNA, 462 bp, Alien to Mouse cDNA
atgagcgggaacctccgtatcaacccatggctgactgcctgcatctgtggggaaaagtcgactcagtgtgggcctgctaaggccgccaacaacaaacgctttcccagggatcaggccagaaagcggctgtattcgccatccccacccatcctgaacacaatgatcctctcccctaaaagttgggtcacgctgcatgttgcgaagaagcaggcccccacgtgttggctgctctccaccgccaacttaaaattccttccatcccagttgcaaccggaggcagatcgaaacttttgtagctctgattaccaccgcactctcccttgtgcgcaggctatcatcacaaatttggagctgaaaatctggacctccaccaaagcgaacagtcccgaacctgtggcgaaagccctggagttcaacacgatagtgccattgtgcaactcagaggaccgctttattgggtag

SEQ ID NO: 81 - DNA, 168 bp, Alien to Mouse cDNA
atgtctcccaacgacattcaggtgattacaggcttgcaccaacgcttgccagtgcttctcaacacccttcgtatgtctgacaaggcattcactctttgctgcaagaagaccaaccctggcagcctgaaaatgcagatgcggaaccgtcacccggatcttcagaaatag

SEQ ID NO: 82 - DNA, 207 bp, Alien to Mouse cDNA
atgatgaagaggcgaactctctctcggatctgcgacatatggacagtgtacggatgcaggaaatgtaaccattacagaaacactattcttcagtccctgtttctcatcttctggattgaaatttgtgaggagcattcccttcattcatcaccgaggcagaccgcctcctcccagttctactcaccgagactcaactcctacgagtaa

SEQ ID NO: 83 - DNA, 144 bp, Alien to Mouse cDNA
atggaccgcccacacatcgtgtccatggcctttttgaactgcgcttcctcagcggccatcttgaagggccataaaatccccctgcccataaagatcctgcgcttcgatccactctctcaaagtactgaatttcctcgggggtag

SEQ ID NO: 84 - DNA, 132 bp, Alien to Mouse cDNA
atgatttttcacctgctgtgctttgctacactcgatgtgaccgtgacgcacacagtggccactgaagcctcgaatggaatgctgatcacgccctctgaagaaatcaccagcaccaggcccgtgatattgtga

SEQ ID NO: 85 - DNA, 192 bp, Alien to Mouse cDNA
atgtgtggcacaggggttagtttaccttctcagataaaacatgaaaacaactttttatttcccgactggacaatgctaaacaagccggaactgtacattggcgggattgaggagaactactgccagtacaagggtcccatctggatcttcagggtggacccgcagtcagaaggccagcgtctgaagttatga

SEQ ID NO: 86 - DNA, 492 bp, Alien to Mouse cDNA
atgatgtttgaggcctgctgcccactcgcggattcgcaggggaagagcaagtccaagggtctgaggaagggagaatctaccccgcttggaggggggcggaagttcctgatgctgtctaccagcctcagcatctactcgtgtattaacatgggccccatctcccttaacgcacacattgatgataacacactccatcagacattcatgtcgcgctcagtgcttgagcggctagttggaacctctcaaaagttcgatacacaccctcatatgtgtgctgcagatgctcagtacacaaagtctagacggtgtgagcaggccttttgggcacccttgtcgcctgcgcttgttttctccatcctctctcaagaaatgggcgacacccccaagaaaaaccggtgtctgaagggtccccagtgcctcaagcgctgttgtcaagagtcctgcctctctggtggctttgtaatctttgacaatccagtctgctacttatga

SEQ ID NO: 87 - DNA, 222 bp, Alien to Mouse cDNA
atgaatgcagaggacatgctggggaaacactgcgcttatgctttttgcacagtccctatcccgaagggagctgtgaacttgaaaaccgagtttgagagtggctgtgcgaagtctgccaacggcaactcccgcaaagacagtgtttcaggtccatgccctaagatgaggcagaagtgggactggggaccccgagaaggagtggctcggacaggagaattctag

SEQ ID NO: 88 - DNA, 150 bp, Alien to Mouse cDNA
atgagagtgagggcacggctgtcaatccccttcaccacgagatccatggccctttgctaccggaagtcgggggacaccggttttgttgtgcagaaggagccccaggatcggtacacgggaaggaaatgtcaacccgtactgatgacctga

SEQ ID NO: 89 - DNA, 297 bp, Alien to Mouse cDNA
atggagaagctgtcctggcgtgctggcctcctccactctcaggatggaataaccagggccgcctacccgggaaaagagcagtcttcccggggccgcaatgcgaccttttggacagctcagcctgactcccgggcggcctcttactcccagctctctgtccagaagtatcgaacaacagcgatgtgcctgcctgtgtccatgtctagtaatctggtctccatggagcagcggttccggcacaagctcatccagtggcggttgtgtctgagaatgtctagtctaaccattatgtcatag

SEQ ID NO: 90 - DNA, 129 bp, Alien to Mouse cDNA
atgtctttgacagattttctttctttctgtgttctgagagtaatggccaaacatctcacagactatagggcctcagctcagcttgggtgctgtgaacagcaggcttctgcatcccgaccggaggaatga

SEQ ID NO: 91 - DNA, 123 bp, Alien to Mouse cDNA
atgacggccttgggggctgcaagttatagccgttctgttgtctatgatggccatccgtctgcgccagagggtggggccaagcgtggcaagcaggtgaagccatggttcaagcaattggaatga

SEQ ID NO: 92 - DNA, 435 bp, Alien to Mouse cDNA
atggtgtggctcctaccccccttaccattgagccactgtaagaatcctttccttcgtaagtgcttcaagtttgagcgctcgtgtgcaggaatttcttgctctgatacgccgccctactcctgccgtcaggccgagagctccacttcatatttttacccattctcaatgaccagaagcaccatgaccatcccagaccaaaccaaaacctgccaggcgtgttctgtgacccggttcccctcccgggaggaaaagaccaagaacctgatgacattctgttacaagatgcatctgcagatggtcggctatccggtcaaagacacgttcctcaaagaggccaaggactctgattcttcagggactgagtttgagctggtgaatgggccacctttttgtgggctcgggattcagttgaactgctgttcccccagtgcctga

SEQ ID NO: 93 - DNA, 198 bp, Alien to Mouse cDNA
atgtccaaggagattcatctgcctgttctgagccgggccggactccctccgagttgtgagaagcttcgaggctccccctctgtgctctccatgacatttgcctaccccctgcccaagcggagccaccaggcaatcgccacggcgtcccgggagctcatgctaaccttggacccctcggccaaaggaccggggtattga

SEQ ID NO: 94 - DNA, 726 bp, Alien to Mouse cDNA
atgcccgcgatggccactggcgcggagtgggcctctgccacacggatatgcgaccgttatgcgacttcccacgtgaggcgcatgagatcaggggcaagactgatcaaacagggagtggagctgatcaagtaccgccccaccacttgcccctacatagccatggatgctcgcgaccttttgcgacacattcggagccccgaatgggaaccctactgctactgtctgacagctatctcaagctcaaagaactatcttctgctgtccgtcagggcccctccattctcgcaaaagaaacgacttcccgtggagtgggtccttcagtgtacccccatctgcaaggcctttcaagggtcaacttcatacaagctgaacatgttctcctcttgcgcgcacactagcgctttgacttcaagggattgcaaaaagtcaatcatgaggcgcaaccattgctacttttatcctttcctggatggagcaggattcccgggggccattacatgcaaaatcagaggatgcattctgggcatgcagaactctccggtgggccgccttaatgggtgctgcaagcagtctgtcagggatgatgagacaaaggcattcctgcagccccgtttggtcgggacgtcaatggtggattatgtgccgctgcaactattctgggagcaagttccgctcctcaagtgttctcttaacccaataagcttgaaagccgcagggacgcagtga

SEQ ID NO: 95 - DNA, 159 bp, Alien to Mouse cDNA
atgtcttatgacttacggtggcttcaccgtggggccacaatcacagccgaaatcatcttatcttgtaagctcccaaaagtgagaatggatttctgctgggtgaagcagtccatggaggccatggtggccatgaaggaccagaaagacgccttttgctga

SEQ ID NO: 96 - DNA, 318 bp, Alien to Mouse cDNA
atgaccagaagctgggccctggtgccaccccacctgttggttggagccgaaacaacccctgtgacttcatatgggtacaaagcgaagagcaacatacgctttgtgttctctgaggcttttgaggctcaacagaggcacgaaagccgttcaaccaaccatgcctgggcccagccagcaggtcgaccggtccatctcattaaggggcaggagaaatctagggaaaatttagatccgagctgtcccaaaccaaagggagcggaccggagtctcacaaaggatggaacaatgaagcaacgatacgacttctacctgccgtaa

SEQ ID NO: 97 - DNA, 732 bp, Alien to Mouse cDNA
atgaagtatgtttcccaggaagcccacctggtctatgtttatatgtatgcggatcactacctcagcagtgtgctgtcttcccaagatgggcgcccctcaaacttcatcacgcgcctgacaaatgcgagtgacaagtggactaacaagacgaagtccatgaaggacagctatcagggtttgtgggagttgcctgggatcctggagctgagagcacctgacatggagctggaacttctgacgaatgggaaagccctgatggcgatccgcatgatcaacatgaagaattccccgcaggatgccaaagaggcctcgtctgcgatcatggccaaagttcccagtttagttgtgccatgctccggctactttgcctggcggcagaagggcttggagcgcaactttgatctgaaaggccaaagtgtcaaatacagaaaaaatacaggtcctggcctgtctccacctcaggtgaggacctcctatcaggaaaacctggggacaccccttctgccaccaattcagatgatgagctacctagtgatttcggacctcccccggaggtctaaacgtgattgcaggcgggcccgtggagtctttgccccacgcgagggactagccaaagaacagggcaaaagcaagctccgcgcagcttacattcacaacaagggtttcgagggcctgactcgtgaacaagtccaggggtatgctgagagctgtgacgttctgccacagcagtag

SEQ ID NO: 98 - DNA, 132 bp, Alien to Mouse cDNA
atgggcacaaagcccttctcactcaagggaaagagctacaagcagcctaacctgaaaatgcaccccctcgtgcctcccttaaacagattcttgtgtcagggtgctgcagttgcagagcggaaaatgcggtaa

SEQ ID NO: 99 - DNA, 441 bp, Alien to Mouse cDNA
atgaatgggctcctgcacacgacatataaggagaagacgtcgtatccgcgtgaggtgtttgggcatagtgcagaaatttcccgcctgtgtcctctgccttccagttccatggcaaccccgccaaatgacgtgaatatggtgatccccctcaaaagacgtgcgctgacgaacacctatgggtctgcttcgattcgtcagatgacgccgatttacaaccctaccgtctctgcctgggtttactcgagccaagaggcactcaagtgtcgttacctgggcttccggcggagaattgaaatgcccttttgttttagtggtgcggccaacagatcctacaacttttctgctaaggaacgcttgggtcacgcacctgcctgtatccgatggcacagatatttatggatgaacttggacatgaaaatgttgactgcccttcgcatctga

SEQ ID NO: 100 - DNA, 70 bp, Oligonucleotides identified according to the present invention as alien to mouse cDNA and useful for hybridization applications.
aaccaatcccatcccaggtgtgcggcgaatcggtcgatctagtcctaattagccggataggaaaacctca

SEQ ID NO: 101 - DNA, 70 bp
aagaacccacgccgtctacatatcgggcacgtgctataacgactcaggagtatttaacgaccgcacggaa

SEQ ID NO: 102 - DNA, 70 bp, Oligonucleotides identified according to the present invention as alien to mouse cDNA and useful for hybridization applications.
acaggtgtcctcaaaccagcctgaaacgttactaggtgaagaatcaccgcggttgtcggtagttaagcga

SEQ ID NO: 103 - DNA, 70 bp, Oligonucleotides identified according to the present invention as alien to mouse cDNA and useful for hybridization applications.
acccgcgtacacagtaggcactctacggcgcgtttagcgttaatcaccaattttgcaatagtcaccagag

SEQ ID NO: 104 - DNA, 70 bp, Oligonucleotides identified according to the present invention as alien to mouse cDNA and useful for hybridization applications.
acggactacctcggccacttcatttggcgacctgcggatattgcttacgaatctcgatcttccggattat

SEQ ID NO: 105 - DNA, 70 bp, Oligonucleotides identified according to the present invention as alien to mouse cDNA and useful for hybridization applications.
agaagtcgtgtgatcgaggtagcactgggatttacgaaaattgccctaccggtatacgctaggccatacc

SEQ ID NO: 106 - DNA, 70 bp, Oligonucleotides identified according to the present invention as alien to mouse cDNA and useful for hybridization applications.
agcccacatatagcccacgcgggtgtcgacaacatatgtcgtatgcgagtaacgttttcgtttgagatgg

SEQ ID NO: 107 - DNA, 70 bp, Oligonucleotides identified according to the present invention as alien to mouse cDNA and useful for hybridization applications.
atactacttttgggtatgctagctacgtagtacccttcaatagccgtcgcttggtctcttgcgcgtcacg

SEQ ID NO: 108 - DNA, 70 bp, Oligonucleotides identified according to the present invention as alien to mouse cDNA and useful for hybridization applications.
catctatctatgtaagttaccggcatgggttatggattcgtggaccgcgatgtgacgtaggggtttccac

SEQ ID NO: 109 - DNA, 70 bp, Oligonucleotides identified according to the present invention as alien to mouse cDNA and useful for hybridization applications.
cattttaccgttaccgggaagcgtgtgtgtctttatttgcgcgtacccagtgttgagaacgacggaacag

SEQ ID NO: 110 - DNA, 70 bp, Oligonucleotides identified according to the present invention as alien to mouse cDNA and useful for hybridization applications.
ccatccgggccataagtttatagtagcgattgttttgcccctaccagcgaatcgcgcccagttagtaatc

SEQ ID NO: 111 - DNA, 70 bp, Oligonucleotides identified according to the present invention as alien to mouse cDNA and useful for hybridization applications.
cccgagcttgcgctagtacgattatgtaccgctatgtcaatttgacgccctcgcactgcggcactttatt

SEQ ID NO: 112 - DNA, 70 bp, Oligonucleotides identified according to the present invention as alien to mouse cDNA and useful for hybridization applications.
ccggctcggtgtcaccgcggaagtacctttgagtatcgcacttatcggctttaacctggacgtaactaaa

SEQ ID NO: 113 - DNA, 70 bp, Oligonucleotides identified according to the present invention as alien to mouse cDNA and useful for hybridization applications.
ccttggatgggtaaattccctcgtctacgcgtaacaactgaacgcgtagcgcgacggtctcaggaaatta

SEQ ID NO: 114 - DNA, 70 bp, Oligonucleotides identified according to the present invention as alien to mouse cDNA and useful for hybridization applications.
cctttccgtgttactcggccggcaaggacgcctcgtaccatctttgatagatgtatttgcgtaaattcgg

SEQ ID NO: 115 - DNA, 70 bp, Oligonucleotides identified according to the present invention as alien to mouse cDNA and useful for hybridization applications.
cgcgaccccgactggtagttgcgcgctcgcattaccgagttcacatcgcatgtactacattagagaaata

SEQ ID NO: 116 - DNA, 70 bp, Oligonucleotides identified according to the present invention as alien to mouse cDNA and useful for hybridization applications.
cggccacaactctcaggacgcatataagacgcggaaaggcatacacgtctacttagagacaccgagactt

SEQ ID NO: 117 - DNA, 70 bp, Oligonucleotides identified according to the present invention as alien to mouse cDNA and useful for hybridization applications.
ctgcttaaccgttccagaggggcgttcgtatcaaaaagggtgcgatttcgatcacgtcgcagtgactcat

SEQ ID NO: 118 - DNA, 70 bp, Oligonucleotides identified according to the present invention as alien to mouse cDNA and useful for hybridization applications.
gaatggcatcaacggcgctgtacatagtcttctcgcctacataatagcgctagttgataggaaccagggg

SEQ ID NO: 119 - DNA, 70 bp, Oligonucleotides identified according to the present invention as alien to mouse cDNA and useful for hybridization applications.
gagctgcacacccgcagacatcatagtgagtgtaatcacgcacgtgaccagttaacccatttcgtggaga

SEQ ID NO: 120 - DNA, 70 bp, Oligonucleotides identified according to the present invention as alien to mouse cDNA and useful for hybridization applications.
gatggattcacgaacgagcacttagtaacgcctggtactgacatcttattgcacgtagtggagagcctgg

SEQ ID NO: 121 - DNA, 70 bp, Oligonucleotides identified according to the present invention as alien to mouse cDNA and useful for hybridization applications.
gcaacgaccagctacctgttaaccgtatatcagagtcgaatgctcgcggtactgttcgaagtactcatcg

SEQ ID NO: 122 - DNA, 70 bp, Oligonucleotides identified according to the present invention as alien to mouse cDNA and useful for hybridization applications.
gcagaattcctaaccatgcaagcgtggcgactcgtctctcgcaaagttctatacgaatcagcgatgggta

SEQ ID NO: 123 - DNA, 70 bp, Oligonucleotides identified according to the present invention as alien to mouse cDNA and useful for hybridization applications.
gccctctcgtcccacgttcgctcgtcttgttgacactactgacgggtatccctctaaatacttctctttt

SEQ ID NO: 124 - DNA, 70 bp, Oligonucleotides identified according to the present invention as alien to mouse cDNA and useful for hybridization applications.
gcctcttcgatggggtccgtctggtcagtaccgacgaaaatgcgacggtagatgtcagaattgattctgt

SEQ ID NO: 125 - DNA, 70 bp, Oligonucleotides identified according to the present invention as alien to mouse cDNA and useful for hybridization applications.
gcgggctcttgtgcaaacttatggggctagtgactcgggtgtagcacgttttgcgaagactaagacagta

SEQ ID NO: 126 - DNA, 70 bp, Oligonucleotides identified according to the present invention as alien to mouse cDNA and useful for hybridization applications.
gcgtctatgacaggtcgggcacttaggcggcgacgcttgatgtttgagtcgcagatattagtttataagg

SEQ ID NO: 127 - DNA, 70 bp, Oligonucleotides identified according to the present invention as alien to mouse cDNA and useful for hybridization applications.
gctatctaacgcggtcttgccaatactacgaatggttgctacaggatatcgagtaccgcaaaatgggggc

SEQ ID NO: 128 - DNA, 70 bp, Oligonucleotides identified according to the present invention as alien to mouse cDNA and useful for hybridization applications.
gggggcaactctccaaccgagcgtgaatccagcgattattatcctactccatactattagcgggtatacg

SEQ ID NO: 129 - DNA, 70 bp, Oligonucleotides identified according to the present invention as alien to mouse cDNA and useful for hybridization applications.
ggtacgaatctcccattgcatggacaaatatagtccacgcattggacgcacccaccgatggctctccaat

SEQ ID NO: 130 - DNA, 70 bp, Oligonucleotides identified according to the present invention as alien to mouse cDNA and useful for hybridization applications.
ggtcgtacccaacctgacacgagatgtcggcgctcgtttcgattggacgatcggatatatgatcaagcaa

SEQ ID NO: 131 - DNA, 70 bp, Oligonucleotides identified according to the present invention as alien to mouse cDNA and useful for hybridization applications.
ggttgttccatgtactcgatactacctaggcatcaggtgtatacgccggtttggatgggcgttcggcaaa

SEQ ID NO: 132 - DNA, 70 bp, Oligonucleotides identified according to the present invention as alien to mouse cDNA and useful for hybridization applications.
gtgccaccccaattagtcttttgtccgggccaagagtacgacaacggggtattttggtactatatcccac

SEQ ID NO: 133 - DNA, 70 bp, Oligonucleotides identified according to the present invention as alien to mouse cDNA and useful for hybridization applications.
gttaagggtctcgaaagatttctactctcgacgtaccgttggcagcgcactaagaacgggtaatgtgctg

SEQ ID NO: 134 - DNA, 70 bp, Oligonucleotides identified according to the present invention as alien to mouse cDNA and useful for hybridization applications.
gttaggcacttgcgcgtcaagcgcgcaaaccctaattacgttctgtccacgcgctagggatattcgtata

SEQ ID NO: 135 - DNA, 70 bp, Oligonucleotides identified according to the present invention as alien to mouse cDNA and useful for hybridization applications.
taagatgcctgacgaaaaagtcccgtgtacccacaacggaaagcgtgatctagatagttcccttagcgcc

SEQ ID NO: 136 - DNA, 70 bp, Oligonucleotides identified according to the present invention as alien to mouse cDNA and useful for hybridization applications.
taattttgggttgtcgaggcataaactggtatgctcgtctcgctcgacgagcggttgaacgcctatcgct

SEQ ID NO: 137 - DNA, 70 bp, Oligonucleotides identified according to the present invention as alien to mouse cDNA and useful for hybridization applications.
tattggccgcggcgctaacttatatcgagagatgtctagtttccccacccgttacatattctacggggag

SEQ ID NO: 138 - DNA, 70 bp, Oligonucleotides identified according to the present invention as alien to mouse cDNA and useful for hybridization applications.
tattttccggtactgagtggaacgacatgaagttggcggtcaggtcgttatttcgcagccacgcaccact

SEQ ID NO: 139 - DNA, 70 bp, Oligonucleotides identified according to the present invention as alien to mouse cDNA and useful for hybridization applications.
tcagatgtcgttattaacgggaaggtatccggttcactatcacggcgattacttcgcgttgcgaaagggc

SEQ ID NO: 140 - DNA, 70 bp, Oligonucleotides identified according to the present invention as alien to mouse cDNA and useful for hybridization applications.
tccggctccgcagacggtttaactcgaaccttaaaagtcgtgtgaagctacttcgagaccatgcgctctt

SEQ ID NO: 141 - DNA, 70 bp, Oligonucleotides identified according to the present invention as alien to mouse cDNA and useful for hybridization applications.
tctgttacccacattgtcaccacttgacaggcgcacggtcgtttgtaaagcgactagctacgcaggtata

SEQ ID NO: 142 - DNA, 70 bp, Oligonucleotides identified according to the present invention as alien to mouse cDNA and useful for hybridization applications.
tggagatgcgaacgttgggagtatcaatccccggtgcaaccccctaatccgacatgccgcaagtatatat

SEQ ID NO: 143 - DNA, 70 bp, Oligonucleotides identified according to the present invention as alien to mouse cDNA and useful for hybridization applications.
tgggcgcctagagccagcatattacaggcgagctgttttcgcgtctctaatgacgtgtacgcgattctat

SEQ ID NO: 144 - DNA, 70 bp, Oligonucleotides identified according to the present invention as alien to mouse cDNA and useful for hybridization applications.
tgtagacagggcgcgattgtatgggacagtttacgcactaaccgactctacaatgtagtgtttgtcgggc

SEQ ID NO: 145 - DNA, 70 bp, Oligonucleotides identified according to the present invention as alien to mouse cDNA and useful for hybridization applications.
ttccgcatgagatcaacgcgtggtcaatacgtgttaagaaccggtcgacgccagctagacctaatgcgtt

SEQ ID NO: 146 - DNA, 70 bp, Oligonucleotides identified according to the present invention as alien to mouse cDNA and useful for hybridization applications.
tttcgactgggggtacaaagctccctatttgccgttcacgaagctacatactggtctagcgcgtgcacaa

SEQ ID NO: 147 - DNA, 47 bp, 10 Ng of alien B was PCR amplified with a forward primer and a reverse primer
ttctaatacgactcactatagggcatctatctatgtcagttaccggc

SEQ ID NO: 148 - DNA, 48 bp, Polymerase sequence
ttttttttttttttttttttttttctaataactgaggtgatttccgac

SEQ ID NO: 149 - DNA, 70 bp, Alien oligonucleotides were first shown to be able to effectivley hybridize with their targets even when included spots containing other oligonucleotides.; identical to SEQ ID NO: 129

SEQ ID NO: 150 - DNA, 23 bp, Anti-alien in spike control concept. Sequences of alien genes designed by linking four 70mer alien sequences together.
ttctaatacgactcactataggg

SEQ ID NO: 151 - DNA, 69 bp, Anti-alien in spike control concept. Sequences of alien genes designed by linking four 70mer alien sequences together.
ccatccgggccatacgtttatagtagcgattgtttgcccctaccagcgaatcgcgcccagttagtaatc

SEQ ID NO: 152 - DNA, 70 bp, Anti-alien in spike control concept. Sequences of alien genes designed by linking four 70mer alien sequences together.
taattttgggttgtcgaggcataaactggtatgctcgtctcgctcgacgagcggttgcacgcctatcgct

SEQ ID NO: 153 - DNA, 70 bp, Anti-alien in spike control concept. Sequences of alien genes designed by linking four 70mer alien sequences together.
gtgccaccccaatttgtcttttgtccgggccaagagtacgacaacggggtattttggtactatatcccac

SEQ ID NO: 154 - DNA, 70 bp, Anti-alien in spike control concept. Sequences of alien genes designed by linking four 70mer alien sequences together.
gcgggctcttgtgcaaacttatggggctggttactcgggtgtagcacgttttgcgaagactacgacagta

SEQ ID NO: 155 - DNA, 19 bp, Anti-alien in spike control concept. Sequences of alien genes designed by linking four 70mer alien sequences together.
aaaaaaaaaaaaaaaaaaa

SEQ ID NO: 156 - DNA, 23 bp, Anti-alien in spike control concept. Sequences of alien genes designed by linking four 70mer alien sequences together.
ttctaatacgactcactataggg

SEQ ID NO: 157 - DNA, 70 bp, Anti-alien in spike control concept. Sequences of alien genes designed by linking four 70mer alien sequences together.
catctatctatgtcagttaccggcatgggttatggattcgtggaccgcgatgtgacgttggggtttccac

SEQ ID NO: 158 - DNA, 70 bp, Anti-alien in spike control concept. Sequences of alien genes designed by linking four 70mer alien sequences together.
tcagatgtcgttattatcgggaaggtatccggttcactatcacggcgattacttcgcgttgcgaaagggc

SEQ ID NO: 159 - DNA, 70 bp, Anti-alien in spike control concept. Sequences of alien genes designed by linking four 70mer alien sequences together.; identical to SEQ ID NO: 152

SEQ ID NO: 160 - DNA, 69 bp, Anti-alien in spike control concept. Sequences of alien genes designed by linking four 70mer alien sequences together.
tccgcatgcgatcaacgcgtggtcaatacgtgtttagaaccggtcgacgccagcttgacctactgcgtt

SEQ ID NO: 161 - DNA, 20 bp, Anti-alien in spike control concept. Sequences of alien genes designed by linking four 70mer alien sequences together.
aaaaaaaaaaaaaaaaaaaa

SEQ ID NO: 162 - DNA, 69 bp, Anti-alien in spike control concept. Sequences of alien genes designed by linking four 70mer alien sequences together.
ccctctcgtcccacgttcgctcgtcttgttgacactactgacgggtatccctctaaatacttctctttt

SEQ ID NO: 163 - DNA, 70 bp, Anti-alien in spike control concept. Sequences of alien genes designed by linking four 70mer alien sequences together.; identical to SEQ ID NO: 133

SEQ ID NO: 164 - DNA, 70 bp, Anti-alien in spike control concept. Sequences of alien genes designed by linking four 70mer alien sequences together.; identical to SEQ ID NO: 138

SEQ ID NO: 165 - DNA, 70 bp, Anti-alien in spike control concept. Sequences of alien genes designed by linking four 70mer alien sequences together.; identical to SEQ ID NO: 116

SEQ ID NO: 166 - DNA, 20 bp, Anti-alien in spike control concept. Sequences of alien genes designed by linking four 70mer alien sequences together.
aaaaaaaaaaaaaaaaaaaa